(19) United States
(12) Patent Application Publication
(10) Pub. No.: US 2011/0179503 A1
(43) Pub. Date: Jul. 21, 2011

(54) PROTEIN PRODUCTION IN A HOST

(75) Inventors: Claude M. Fauquet, Saint Louis, MO (US); Mohammad Abhary, Saint Louis, MO (US)

(73) Assignee: Donald Danforth Plant Science Center

(21) Appl. No.: 13/055,304

(22) PCT Filed: Jul. 21, 2009
(86) PCT No.: PCT/US09/51295

§ 371 Date: Apr. 7, 2011

Related U.S. Application Data
(60) Provisional application No. 61/082,506, filed on Jul. 21, 2008.

(51) Int. Cl. A01K 67/00; C12N 15/63; C12N 1/00; C12N 5/10; A01H 5/00; A01H 5/10; C12P 21/00

(52) U.S. Cl. 800/4; 435/320.1; 435/243; 435/419; 435/325; 800/298; 800/13; 435/69.1

(57) ABSTRACT

Recombinant DNA constructs that provide for expression of desired target proteins as protein bodies in various host cells, tissues, and organisms are disclosed. Such recombinant DNA constructs contain a heterologous polynucleotide comprising an ER luminal folding chaperone interacting domain (CID), a sequence that codes for a trans-membrane domain (TMD); and a sequence that codes for a protein-protein interaction domain (PPID) that are operably linked to a sequence encoding the desired target protein. Proteins obtained from the constructs, host cells containing the constructs, and methods for obtaining desired target proteins encoded by the constructs are also disclosed.

### CROSS-REFERENCE TO RELATED APPLICATIONS

This application claims the benefit of U.S. Provisional Patent Application No. 61/082,506, filed Jul. 21, 2008, and incorporated herein by reference in its entirety.

### TECHNICAL FIELD

The present invention relates to the field of genetics. More specifically, the present invention relates to constructs and methods of using the constructs to modify plants to produce a protein of interest of superior quality, quantity, stability, and/or other desirable physicochemical property.

### INCORPORATION OF SEQUENCE LISTING

A computer readable form of the Sequence Listing is provided herein, contained in the file named “47004-84158_ST25.txt,” which is 36301 bytes (as measured in MS-DOS), and is herein incorporated by reference. This Sequence Listing consists of SEQ ID NOs: 1-21.

### BACKGROUND

Plants represent a major economical system for large-scale production of proteins and recombinant proteins that are important in pharmaceutical and industrial uses (Ma et al. Nature Reviews Genetics 4: 794-805, 2003) with many advantages over other systems; the low production cost and the fact that plants are not susceptible to human pathogens which can contaminate the product as in mammalian expression system, and the ability of targeting the expression to specific plant tissues such as roots, fruits, leaves, or seeds, make the delivery process of a recombinant product to human consumer easier and safer than other expression systems.

Legumes and cereal grain plants accumulate storage proteins in the ER during development and maturation of their seeds, building ER spherical structures packed with proteins called Protein Bodies (PB) (Herman and Larkins, Plant Cell, 11: 601-614,1999). Protein classes that can form PB's contain a signal peptide and/or mRNA involved in targeting to specific subdomains of the ER membrane and have the ability to interact with each other to stabilize tertiary structure and the accumulation process, avoiding degradation and breakdown. That structure involves hydrogen, Van der Waals, ionic and disulfide bonds in the polypeptide, and also an interaction between the individual polypeptides to form protein aggregates within the ER. The ability of legumes and cereal seeds to form PB is not restricted to the plant species, but rather it is coded within the proteins composing the PB. For example, expressing maize zeins in tobacco cells results in the formation of the same PB, indicating that those expressed proteins possess a signal that triggers PB formation in cells that naturally do not contain PB.

Maize and rice store some of their seed proteins in the ER as PB to be used later during germination as a nitrogen source. In the case of synthetic PB such as Zeolin (Mainieri et al., 2004, Plant Physiology, 136:3447-3456), degradation is not reported to take place in these cells before cell death. The process of PB formation and the signaling domains on Zeolin are poorly understood. Various changes in the peptide sequence of Zeolin can block protein body formation, limiting the utility of the Zeolin system.

Technologies exist for creation of synthetic PBs based on the fusion of a plant seed storage protein domain with a heterologous protein of interest (WO 2004/003207) to increase the stability and accumulation of recombinant proteins in higher plants. These storage proteins are specific to plant seeds wherein they accumulate stably in natural PBs (Galili et al., 1993, Trends Cell Biol 3:437-442) following insertion into the lumen of the ER via a signal peptide and assembly into ER-derived protein bodies (ER-PBs) (Okita et al., 1996 Annu. Rev. Plant Physiol Mol. Biol. 47:327-350; Herman et al., 1999 Plant Cell 11:601-613; Sanderfoot et al., 1999 Plant Cell 11:629-642).

Full-length recombinant storage proteins have also been observed to assemble into PB-like organelles in non-plant host systems as Xenopus oocytes following injection of the corresponding mRNAs. This system has been used as a model to study the targeting properties of these storage proteins (Simon et al., 1990, Plant Cell 2:941-950; Altschuler et al., 1993, Plant Cell 5:443-450; Torrent et al., 1994, Planta 192:512-518) and to test the possibility of modifying the 19 kDa.alpha.-zein, a maize prolamin, by introducing the essential amino acids lysine and tryptophan into its sequence, without altering its stability (Wallace et al, 1988, Science 240:662-664).

Zeins, the complex group of maize prolamins, have also been produced recombinantly in yeast. Coraggio et al. (1988, Eur J Cell Biol 47:165-172), expressed native and modified .alpha.-zeins in yeast to study targeting determinants of this protein. Kim et al. (2002, Plant Cell 14: 655-672) studied the possible .alpha.-, .beta.-, .gamma.- and .delta.-zein interactions that could lead to protein body formation. To address this question, they transformed yeast cells with cDNAs encoding these proteins. In addition, those authors constructed zein-GFP fusion proteins to determine the subcellular localization of zein proteins in yeast cells but did not observe formation of dense, concentrated structures characteristic of bona fide PBs. It is worth noting that Kim et al. (2002, Plant Cell 14: 655-672) concluded that yeast is a poor model for the study of zein interactions because zeins accumulated very poorly in transformed yeast. Yeast has also been used as a model to study the mechanisms that control the transport and deposition of gliadin storage proteins in wheat (Rosenberg et al., 1993, Plant Physiol 102:61-69).

A particular case involving use of maize zeins as a signal to trigger PB formation is “Zeolin” (Mainieri et al., 2004, Plant Physiology, 136:3447-3456). As previously described, Zeolin is a chimeric protein composed of Phaseolin, the major storage protein in white beans (Phaseolis vulgaris), and a portion of gamma zein from maize. The resulting fusion polypeptide has the ability to form PB in tobacco cells as well as in alfalfa. Unfortunately, when the phaseolin part of Zeolin is substituted with another protein, PB will no longer form. Thus, the utility of the Zeolin-based system for expressing foreign or target proteins as PB is limited

What is needed in the art is a flexible system for producing a target protein in a host of interest in a desirable form, for example, in a PB. Moreover, it would be desirable if a target protein could be produced in superior quality, quantity, stability, and/or other desirable physicochemical property.

### SUMMARY OF THE INVENTION

It has now been discovered that by combining coding sequences for moieties as taught herein, a flexible system can be achieved for producing a protein of interest of superior quality, quantity, stability, and/or other desirable physicochemical property. Also provided herein are associated methods for obtaining or isolating a protein of interest.

In certain embodiments, a construct of the present invention comprises a promoter functional in one or more tissues in a host and a heterologous polynucleotide comprising:

a) a sequence that codes for a moiety that interacts with an ER luminal folding chaperone (“CID” for chaperone interacting domain);

b) a sequence that codes for trans-membrane domain (TMD); and

c) a sequence that codes for a protein-protein interaction domain (PPID);

wherein the heterologous polynucleotide is operably linked to the promoter with the proviso that the coding sequences for the CID, TMD, and PPID are not contained elsewhere in a contiguous polynucleotide coding for a naturally occurring protein or a synthetic protein known in the art. For example, the heterologous polynucleotide does not encode a naturally occurring member of any of the families of zeins, 7S or 11S globulin, vicilin, legumin, prolamin, gliadin, or encode Zeolin. In certain embodiments, constructs comprising a promoter functional in a host and a heterologous polynucleotide comprising:

(a) a sequence that codes for a moiety that interacts with an ER luminal folding chaperone (“CID” for chaperone interacting domain (CID));

(b) a sequence that codes for a trans-membrane-coding domain (TMD); and

(c) a sequence that codes for a protein-protein interaction domain (PPID),

wherein the heterologous polynucleotide is operably linked to the promoter are provided. In certain embodiments, the heterologous polynucleotide of the construct does not encode a naturally occurring form of a protein sequence comprising all of said sequences (a), (b), and (c). In certain embodiments, the heterologous polynucleotide of the construct comprises a site for operable insertion of a sequence that encodes a polypeptide. In certain embodiments, the heterologous polynucleotide of the construct comprises in 5′ to 3′ order: (i) the sequence of (a); (ii) the site for operable insertion of a sequence; (iii) the sequence of (b); and (iv) the sequence of (c), wherein insertion of a sequence that encodes a polypeptide in said site for operable insertion provides for operable linkage of said sequence encoding said polypeptide to (i), (iii), and (iv). In certain embodiments, the heterologous polynucleotide comprises in 5′ to 3′ order: (i) said sequence of (a); (ii) said sequence of (b); (iii) said sequence of (c); and (iv) said site for operable insertion of a sequence, wherein insertion of a sequence that encodes a polypeptide in said site for operable insertion provides for operable linkage of said sequence encoding said polypeptide to (i), (ii), and (iii). In certain embodiments, the heterologous polynucleotide of the construct further comprises a sequence coding for a target protein that is operably linked to: i) said sequence that codes for a chaperone interacting domain (CID); ii) said sequence that codes for a trans-membrane-domain (TMD); and iii) said sequence that codes for a protein-protein interaction domain (PPID). In certain embodiments, the heterologous polynucleotide of the construct comprises in 5′ to 3′ order: (i) the sequence of (a); (ii) the sequence encoding a target protein; (iii) the sequence of (b); and (iv) said sequence of (c), wherein (i), (ii), (iii), and (iv) are operably linked. In certain embodiments, the heterologous polynucleotide comprises in 5′ to 3′ order: (i) the sequence of (a); (ii) the sequence of (b); (iii) the sequence of (c); and (iv) the sequence encoding a target protein; wherein (i), (ii), (iii), and (iv) are operably linked. In certain embodiments, the target protein inserted in the construct is BHL8, Sporamine, or a Green Fluorescent Protein (GFP). In certain embodiments, the target protein of the construct does not in a naturally occurring form comprise any one of or all of: (a) a sequence that codes for a moiety that interacts with an ER luminal folding chaperone interacting domain (CID); (b) a sequence that codes for a trans-membrane-coding domain (TMD); and (c) a sequence that codes for a protein-protein interaction domain (PPID). In certain embodiments of any of the above constructs, the construct can further comprise at least one operably linked regulatory element selected from a 3′ UTR translational enhancer domain and a terminator.

In certain embodiments, one or more members selected from the group of sequences coding for a CID, a TMD, a PPID, and a target protein is heterologous to a second member of the same group of sequences. By “heterologous to” it is meant that two such members are not found on a contiguous polynucleotide coding for a natural or a synthetic protein. By way of an exemplary embodiment, CID, TMD, and PPID useful in the present invention are not used with a natural or synthetic polynucleotide containing the target protein of the present invention.

Optionally, the heterologous polynucleotide further comprises a sequence coding for a target protein.

In certain embodiments, hosts transformed with optional constructs of the present invention, a target protein is produced and localized to the ER or to ER-derived vesicles.

Optionally, the target protein is produced as a fusion protein containing one or more of peptide sequences coded by TMD, PPID, or CID sequences.

In other embodiments, the target protein is produced by a host transformed with a construct according to the present invention is interrupted by peptide sequences coded by one or more of TMD, PPID, or CID.

Also provided are hosts transformed with any of the aforementioned constructs of the invention. In certain embodiments, the host transformed with a construct provided herein accumulates the target protein in PB. In certain embodiments, the host transformed with a construct provided herein comprises a target protein that is substantially non-glycosylated. In certain embodiments, the host transformed with any of the aforementioned constructs is a cell, a tissue, an organ, or non-human organism. In certain embodiments, the host transformed with a construct provided herein is a monocot plant, a monocot plant cell, a monocot plant seed, a dicot plant, a dicot plant seed, a dicot plant cell, a non-human animal, a procaryotic cell, or a eukaryotic cell.

Proteins encoded by the heterologous polynucleotide of a construct of the invention are also provided herein. In certain embodiments, the protein of the invention provided herein comprises at least one of or all of: (a) ER luminal folding chaperone interacting domain (CID); (b) a trans-membrane-coding domain (TMD); and (c) protein-protein interaction domain (PPID), where the naturally occurring form of the target protein does not contain any one of an (a) ER luminal folding chaperone interacting domain (CID), (b) a trans-membrane-coding domain (TMD); or a (c) protein-protein interaction domain (PPID).

Also provided are methods of producing a target protein comprising the step of growing a host comprising a construct of the invention under conditions that provide for expression of a protein encoded by the heterologous polynucleotide of the construct. In certain embodiments, the target protein produced by the method is a target protein that does not form protein bodies in its naturally occurring form. In certain embodiments, the methods can further comprise purification of the protein encoded by the heterologous polynucleotide or purification of a portion of the protein encoded by the heterologous polynucleotide. In certain embodiments, purification can comprise a step wherein the protein or a portion thereof is recovered as a protein body. In certain embodiments, protein bodies are recovered by density centrifugation. In certain embodiments, methods involve purification further comprises recovery of said protein or a portion thereof from said protein body by treating said protein body with an aqueous buffer containing a detergent and a reducing agent.

Also provided herein are a host genomic DNA comprising any of the aforementioned constructs.

Also provided herein are processed products of a plant storage organ comprising a target protein encoded by the heterologous polynucleotide of any one of the aforementioned constructs. In certain embodiments, the processed product is a flour, a meal, a juice, or a homogenate. In certain embodiments, the plant storage organ is selected from the group consisting of a root, a tuber, a flesh of a fruit, and a seed.

### BRIEF DESCRIPTION OF THE DRAWINGS

FIG. 1 shows a construct of the present invention with an optional promoter.

FIG. 2 shows the cloning of Zeolin in a binary vector.

FIG. 3 shows the sequence of Zeolin (SEQ ID NO: 13).

FIG. 4 shows cassava transformation with a Zeolin expression construct.

FIG. 5 shows the detection of Zeolin in Cassava Roots.

FIG. 6 shows confocal microscopy of Zeolin in expressed in Cassava roots.

FIG. 7 shows scanning electron microscopy (SEM) of purified protein bodies from transgenic (A) BY2 cells and (B) Cassava storage parenchyma root cells.

FIG. 8 shows temporal expression of Zeolin in root tissues.

FIG. 9 shows accumulation of total protein content over time in root tissues.

FIG. 10 shows the general structure of a protein body shuttle vector (PBSV) cassette constructs of the present invention.

FIG. 11 shows a general structure of an exemplary PBSV construct of the present invention.

FIG. 12 shows the sequence of the PBSV-2 construct of the present invention (SEQ ID NO:9).

FIG. 13 shows vector NTI maps of the PBSV constructs.

FIG. 14 shows transformed BY2 cells.

FIG. 15 shows the detection of transgenic events by DNA Hybridization.

FIG. 16 shows quantification of RNA transcripts by RT-PCR.

FIG. 17 shows Northern blot analysis of transgenic BY2 cells.

FIG. 18 shows Western dot blots of proteins produced in transgenic BY2 cells.

FIG. 19 shows Western blots of proteins produced in transgenic BY2 cells.

FIG. 20 shows quantification of protein accumulation proteins produced in transgenic BY2 cells. Allergenicity information was obtained from the database made available on the World Wide Web at http://www.allergenonline.org that is maintained by the University of Nebraska in Lincoln, Nebr., USA.

FIG. 21 shows subcellular localization of transgenic proteins in transformed BY2 Cells.

FIG. 22 shows subcellular localization of transgenic proteins in transformed BY2 Cells.

FIG. 23 shows subcellular localization of target protein in a mutant construct (PBSV-1 ml; amino acid sequence provided as SEQ ID NO: 11; DNA sequence provided as SEQ ID NO:16).

FIG. 24 shows subcellular localization of a transgenic protein in transformed BY2 Cells deficient in BIP.

FIG. 25 shows the biochemical characterization of a transgenic protein in transformed BY2 Cells deficient in BIP by SDS-PAGE.

FIGS. 26a, b: FIG. 26a shows PBSV-1 and comparator constructs; 26b shows in-situ RNA hybridization of BY2 cells transformed with PBSV-1 and a comparator constructs.

FIG. 27 shows subcellular localization of the target protein of PBSV-3, 5 days post transformation in BY2 cells.

FIG. 28 shows the PBSV-4 vector.

FIG. 29 shows the protein bodies formed by expression of sporamine encoded by the PBSV-4 vector.

FIG. 30 shows a gel electrophoretic analysis of protein bodies purified by ficoll gradient centrifugation.

### DETAILED DESCRIPTION OF THE INVENTION

As used herein, the following abbreviations, terms, and phrases apply.

“CID” means a sequence that interacts with the ER luminal protein chaperon (e.g. BIP), a resident of the ER, which helps to give the nascent polypeptide its three dimensional structure.

“Fusion protein” designates a polypeptide comprising a target protein synthesized from a present construct (e.g. by a transformed host) that contains at least two amino acid residue sequences not normally found linked together in nature that are operatively linked together end-to-end (head-to-tail) by a peptide bond between their respective carboxy- and amino-terminal amino acid residues. The fusion proteins of the present invention can be chimeras of the target protein with the moieties synthesized from any of the CID, PPID, and TMD coding domains.

The phrase “operable insertion” as used herein refers to the insertion of one or more additional nucleic acid sequences into a nucleic acid construct so that the additional sequence(s) are operably linked to at least one other sequence in the construct.

The phrase “operably linked” as used herein refers to the joining of nucleic acid sequences such that one sequence can provide a function to a linked sequence. In the context of a promoter, “operably linked” means that the promoter is connected to a sequence of interest such that the transcription of that sequence of interest is controlled and regulated by that promoter. When the sequence of interest encodes a protein and when expression of that protein is desired, “operably linked” means that the promoter is linked to the sequence in such a way that the resulting transcript will be efficiently translated. If the linkage of the promoter to the coding sequence is a transcriptional fusion and expression of the encoded protein is desired, the linkage is made so that the first translational initiation codon in the resulting transcript is the initiation codon of the coding sequence. Alternatively, if the linkage of the promoter to the coding sequence is a translational fusion and expression of the encoded protein is desired, the linkage is made so that the first translational initiation codon contained in the 5′ untranslated sequence associated with the promoter is linked such that the resulting translation product is in frame with the translational open reading frame that encodes the protein desired. Nucleic acid sequences that can be operably linked include, but are not limited to, sequences that provide gene expression functions (i.e., gene expression elements such as promoters, 5′ untranslated regions, introns, protein coding regions, 3′ untranslated regions, polyadenylation sites, and/or transcriptional terminators), sequences that provide DNA transfer and/or integration functions (i.e., site specific recombinase recognition sites, integrase recognition sites), sequences that provide for selective functions (i.e., antibiotic resistance markers, biosynthetic genes), sequences that provide scoreable marker functions (i.e., reporter genes), sequences that facilitate in vitro or in vivo manipulations of the sequences (i.e., polylinker sequences, site specific recombination sequences, homologous recombination sequences), and sequences that provide replication functions (i.e., bacterial origins of replication, autonomous replication sequences, centromeric sequences). In the context of nucleic acid sequences that encode proteins, operably linked means that such sequences are joined so as to provide for translation of the desired protein sequences and preservation of the desired function. Thus, operable linkage of a CID, TMD, and PPID encoding sequence to a target protein encoding sequence provides for a composite protein that both comprises and retains the desired functional attributes of the respective CID, TMD, PPID, and target protein sequences.

“PB” means protein body which is a morphologically recognizable body that contains concentrated amounts of the target protein. PB is meant to embrace bodies naturally made in, for example, seeds of plants. It is also meant to embrace bodies that might not otherwise exist naturally in the host but that, due to technical features of constructs of the present invention, are formed therein. Protein bodies formed in hosts transformed by constructs of the present invention generally contain the target protein at a high concentration to the seclusion of other cellular proteins.

“PPID” means a sequence that codes for a protein-protein interaction domain.

As used herein in the context of methods for purification of a target protein, the term “purification” refers any technique that provides for any degree of separation of a target protein from any other material in a host or host derived composition that comprise the target protein.

“Target protein” means a protein produced by a host transformed by a present construct as coded for by the target protein coding sequence. It should be understood that the term “target protein” is also meant to include a target protein fusion protein (e.g. produced by the host comprising the peptide coded by the target protein coding sequence fused to one or more of peptide sequences coded by TMD, PPID, or CID sequences). “Target protein” is also meant to include a protein comprising the peptide coded by the target protein coding sequence interrupted by one or more peptide sequences coded by TMD, PPID, or CID sequences. “Target protein” is also meant to include a protein comprising the peptide coded by the target protein coding sequence interrupted by one or more peptide sequences coded by TMD, PPID, or CID sequences and fused to one or more peptide sequences coded by TMD, PPID, or CID sequences.

“TMD” means a sequence that codes for a trans-membrane domain.

To the extent to which any of the preceding definitions is inconsistent with definitions provided in any patent or non-patent reference incorporated herein or in any reference found elsewhere, it is understood that the preceding definition will be used herein.

Architecture

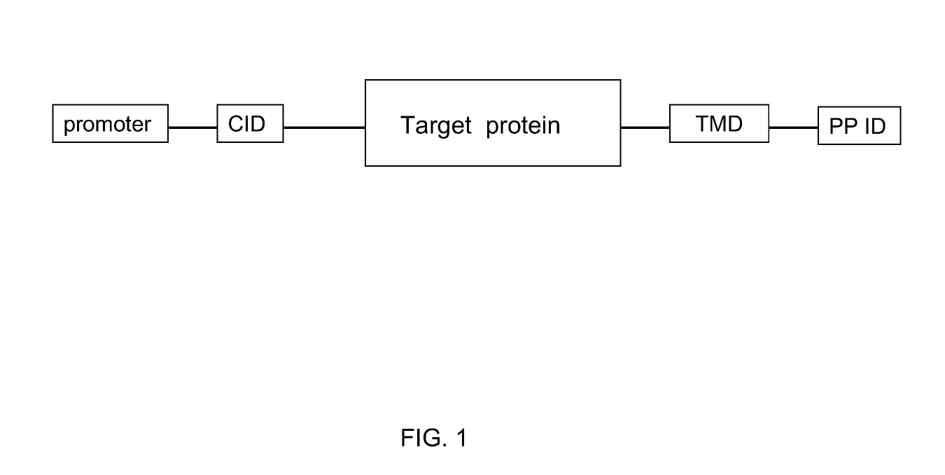

In certain exemplary and non-limiting embodiments, one construct class of the present invention is illustrated in FIG. 1. It should be noted that there are optional relative positions for the sequence domains of the present invention.

In certain exemplary and non-limiting embodiments, the target protein sequence can instead or additionally be interrupted by one or more of a TMD, PPID, and CID sequences.

In certain exemplary and non-limiting embodiments, one or more of the TMD, PPID, and CID encoding sequences can instead or additionally be 5′ to target protein sequence.

In certain exemplary and non-limiting embodiments, one or more of the TMD, PPID, and CID encoding sequences can instead or additionally be provided by the target protein coding sequence.

Depending upon the optional architecture of constructs of the present invention, a host transformed with a construct produces a target protein fused to one or more of peptide sequences coded by TMD, PPID, or CID sequences.

In other embodiments, the target protein produced by a host transformed with a construct according to the present invention is interrupted by peptide sequences coded by one or more of TMD, PPID, or CID.

It is to be understood that the domains that code for TMD, PPID, CID, and target protein are arranged and linked such that the domains are expressed in-frame when transformed into a host of the present invention.

Delivery to ER-Derived Vesicles

Constructs of the present invention combine sequences that modulate one or more of synthesis, intracellular transport, and cellular localization of the target protein. In some embodiments, the target protein accumulates in PBs.

In some embodiments, the PB's are ER-derived protein bodies that do not transit the Golgi complex.

In other embodiments, the PB's are ER-derived vesicles generally devoid of vacuolar enzymes (e.g., as compared to vacuoles).

In other embodiments, the target protein is localized primarily within the lumen of the ER.

In other embodiments, the target protein is localized primarily within regions of the ER as a dense accumulation of target protein including target protein aggregates.

In other embodiments, the target protein is localized primarily to structures generally recognized as PB's that otherwise naturally occur in a host.

In other embodiments, the target protein is localized primarily to protein bodies that are enclosed in a membrane.

In other embodiments, the PB's are present in a generally spherical form having a diameter of about 0.5 to about 3 microns or in PB's of amorphous shape.

In other embodiments, the PB's are present at a predetermined density that can differ among target protein, but is predictable across hosts for a particular target protein being prepared. That predetermined density of the PB's is typically greater than that of substantially all of the endogenous host cell proteins present in a host homogenate and is typically about 1.1 to about 1.35 g/ml. The high density of PB's produced by the present invention is due to the general ability of the target protein to self-assemble and accumulate into ordered aggregates associated with membranes.

Promoters

A polynucleotide of the present invention may comprise a promoter functional in a host. The promoter can be selected according to the expression desired in the host. The promoter can be tissue or organ specific. For example, it can be for root or seed specific expression. A promoter can be one that can be regulated by intrinsic or extrinsic modulators. The promoter can be selected to be functional in all host cells.

One manner of achieving storage organ expression is to use a promoter that expresses its controlled gene in one or more preselected or predetermined non-photosynthetic plant organs. Expression in one or more preselected storage organs with little or no expression in other organs such as roots, seed or fruit versus leaves or stems is referred to herein as enhanced or preferential expression. An exemplary promoter that directs expression in one or more preselected organs as compared to another organ at a ratio of at least 5:1 is defined herein as an organ-enhanced promoter. Expression in substantially only one storage organ and substantially no expression in other storage organs is referred to as organ-specific expression; i.e., a ratio of expression products in a storage organ relative to another of about 100:1 or greater indicates organ specificity. Storage organ-specific promoters are thus members of the class of storage organ-enhanced promoters.

Exemplary plant storage organs include, but are not limited to, the roots of carrots, taro or manioc, potato tubers, and the meat of fruit such as red guava, passion fruit, mango, papaya, tomato, avocado, cherry, tangerine, mandarin, palm, melons such cantaloupe and watermelons and other fleshy fruits such as squash, cucumbers, mangos, apricots, peaches, as well as the seeds of maize (corn), soybeans, rice, oil seed rape and the like.

The CaMV 35S promoter is normally deemed to be a constitutive promoter. However, research has shown that a 21-bp region of the CaMV 35S promoter, when operatively linked into another, heterologous usual green tissue promoter, the rbcS-3A promoter, can cause the resulting chimeric promoter to become a root-enhanced promoter. That 21-bp sequence is disclosed in U.S. Pat. No. 5,023,179. The chimeric rbcS-3A promoter containing the 21-bp insert of U.S. Pat. No. 5,023,179 is a useful root-enhanced promoter herein.

A similar root-enhanced promoter, which includes the above 21-bp segment, is the −90 to +8 region of the CAMV 35S promoter itself. U.S. Pat. No. 5,110,732 discloses that that truncated CaMV 35S promoter provides enhanced expression in roots and the radicle of seed, a tissue destined to become a root. That promoter is also useful herein.

Another useful root-enhanced promoter is the −1616 to −1 promoter of the oil seed rape (Brassica napus L.) gene disclosed in PCT/GB92/00416 (WO 91/13922 published Sep. 19, 1991). E. coli DH5-alpha harboring plasmid pRlambdaS4 and bacteriophage lambda.beta.1 that contain this promoter were deposited at the National Collection of Industrial and Marine Bacteria, Aberdeen, GB on Mar. 8, 1990 and have accession numbers NCIMB40265 and NCIMB40266. A useful portion of this promoter can be obtained as a 1.0 kb fragment by cleavage of the plasmid with HaeIII.

A preferred root-enhanced promoter is the mannopine synthase (mas) promoter present in plasmid pKan2 described by DiRita and Gelvin (1987) Mol. Gen. Genet, 207:233-241. This promoter is removable from its plasmid pKan2 as a XbaI-XbalI fragment.

A preferred mannopine synthase root-enhanced promoter is comprised of the core mannopine synthase (mas) promoter region up to position −138 and the mannopine synthase activator from −318 to −213, and is collectively referred to as AmasPmas. This promoter has been found to increase production in tobacco roots about 10- to about 100-fold compared to leaf expression levels.

Another root specific promoter is the about 500 bp 5′ flanking sequence accompanying the hydroxyproline-rich glycopeprotein gene, HRGPnt3, expressed during lateral root initiation and reported by Keller et al. (1989) Genes Dev., 3:1639-1646. Another preferred root-specific promoter is present in the about -636 to −1 5′ flanking region of the tobacco root-specific gene TORBF reported by Yamamoto et al. (1991) Plant Cell, 3:371-381. The cis-acting elements regulating expression are more specifically located by those authors in the region from about −636 to about −299 5′ from the transcription initiation site. Yamamoto et al. reported steady state mRNA production from the TORBF gene in roots, but not in leaves, shoot meristems or stems.

Still another useful storage organ-specific promoter is derived from the 5′ and 3′ flanking regions of the fruit-ripening gene E8 of the tomato, Lycopersicon esculentum. These regions and their cDNA sequences are illustrated and discussed in Deikman et al. (1988) EMBO J., 7(11):3315-3320 and (1992) Plant Physiol., 100:2013-2017.

Three regions are located in the 2181 bp of the 5′ flanking sequence of the gene and a 522 bp sequence 31 to the poly (A) addition site appeared to control expression of the E8 gene. One region from −2181 to −1088 is required for activation of E8 gene transcription in unripe fruit by ethylene and also contributes to transcription during ripening. Two further regions, −1088 to −863 and −409 to −263, are unable to confer ethylene responsiveness in unripe fruit but are sufficient for E8 gene expression during ripening.

The maize sucrose synthase-1 (Sh) promoter that, in corn, expresses its controlled enzyme at high levels in endosperm, at much reduced levels in roots, and not in green tissues or pollen has been reported to express a chimeric reporter gene, .beta.-glucuronidase (GUS), specifically in tobacco phloem cells that are abundant in stems and roots. Yang et al. (1990) Proc. Natl. Acad. Sci., U.S.A., 87:4144-4148. This promoter is thus useful for plant organs such as fleshy fruits like melons, e.g. cantaloupe, or seeds that contain endosperm and for roots that have high levels of phloem cells.

Another exemplary tissue-specific promoter is the lectin promoter, which is specific for seed tissue. The lectin protein in soybean seeds is encoded by a single gene (Le1) that is only expressed during seed maturation and accounts for about 2 to about 5 percent of total seed mRNA. The lectin gene and seed-specific promoter have been fully characterized and used to direct seed specific expression in transgenic tobacco plants. See, e.g., Vodkin et al. (1983) Cell, 34:1023 and Lindstrom et al. (1990) Developmental Genetics, 11:160.

A particularly preferred tuber-specific expression promoter is the 5′ flanking region of the potato patatin gene. Use of this promoter is described in Twell et al. (1987) Plant Mol. Biol., 9:365-375. This promoter is present in an about 406 bp fragment of bacteriophage LPOTI. The LPOTI promoter has regions of over 90 percent homology with four other patatin promoters and about 95 percent homology over all 400 bases with patatin promoter PGT5. Each of these promoters is useful herein. See, also, Wenzler et al. (1989) Plant Mol. Biol., 12:41-50.

Still further higher plant organ-enhanced and organ-specific promoters are disclosed in Benfey et al. (1988) Science, 244:174-181.

Optionally, each of the promoter sequences utilized is substantially unaffected by the amount of PB's in the cell. As used herein, the term “substantially unaffected” means that the promoter is not responsive to direct feedback control (inhibition) by the PB's accumulated in transformed cells or transgenic host.

TMD Sequences

The TMD of the present invention can be any sequence that codes for a stretch of at least 10 amino acid residues where a majority of the residues are hydrophobic in nature and can form an alpha helix. By way of example, SEQ ID NO:1, derived from β-Zein, is useful as a trans-membrane-coding sequence of the present invention. In certain embodiments, the TMD can comprise a variant of SEQ ID NO:1 that includes but is not limited to, conservatively substituted variants of SEQ ID NO:1, or protein domains with at least 80%, at least 85%, at least 90%, at least 95%, or at least 98% sequence identity to SEQ ID NO:1.

A TMD sequence useful according to the present invention can now be identified based upon a hydrophobicity plot.

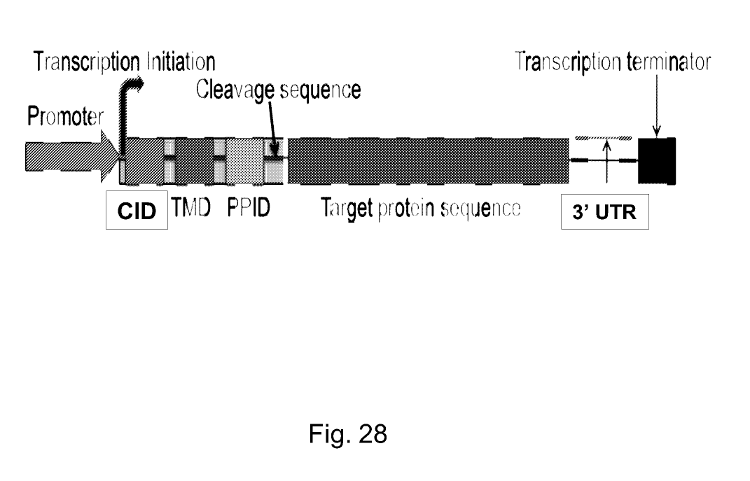

Without seeking to be limited by theory, it is believed that the TMD of the present invention provides stability by anchoring the target protein to the membrane surrounding the PB in the ER or ER derived vesicles. The TMD sequence can be located at the carboxyl terminal end of the target protein as illustrated in FIG. 1 or placed after the CID and amino-terminal to an optional target protein sequence as illustrated in FIG. 28.

PPID

A sequence coding for a protein-protein interaction domain (PPID) of the present invention can be any sequence that results in protein aggregation. By way of example, protein-protein interaction is known to occur through disulfide bonds, especially in hydrophilic regions of the fusions. By way of example, a PPID can code for a peptide sequence that has 4 or 5 or 6 or 7 or 8 or more cysteine residues. A PPID can contain cysteine residues distributed throughout a sequence coding for more than about 10 amino acid residues or more than about 20 amino acid residues or more than about 30 amino acid residues. By way of example, SEQ ID NO:2 derived from beta-Zein, is useful as such a sequence. In certain embodiments, the PPID can comprise a variant of SEQ ID NO:2 that includes but is not limited to, conservatively substituted variants of SEQ ID NO:2, or protein domains with at least 80%, at least 85%, at least 90%, at least 95%, or at least 98% sequence identity to SEQ ID NO:2.

Optionally, a PPID may be derived from a peptide sequence that is capable of self-aggregation when present outside of a fusion protein of the present invention (i.e., an isolated PPID). For example, amyloid beta sequences (including subsequences thereof) are known to aggregate and form layers of beta sheets. Other sequences known to support scaffold formation, including sequences with sufficient cysteine content, can also form the PPID.

Optionally, the PPID forms a tail, the length of which is selected with respect the size or bulkiness of the target protein or the rest of the fusion protein in order to engineer a protein body of a specific size or density. In one embodiment, the interaction of PPID tails promotes spherical aggregation of the fusion proteins in a manner similar to micelle formation of phospholipids.

Optionally, the fusion protein may align to form a sphere, wherein the “shell” of the sphere is comprised of packed proteins with their PPID tails directed inward. Accordingly, larger proteins may require a relatively longer tail to form an aggregate or sphere of a desired size, density, and/or stability. A plurality of spheres can optionally be further aggregated together by incorporating one or more additional PPIPs on the portion of the protein that is exposed on sphere such that spheres interact with each other to form larger aggregates.

Optionally, the location, size, and type of PPID can be varied to engineer specific PPID interactions which form protein bodies of desired geometries, density, and/or stability (i.e. the puzzle approach wherein each fusion protein forms one piece of the puzzle). Several PPIDs can be incorporated such that each interacts with a specific PPID on one or more other fusion proteins, which may be identical fusion proteins, or distinct fusion proteins also engineered to interact in a specific manner to produce the desired geometry, density, and/or stability.

Yeast 2-hybrid system can be used now by the skilled artisan to identify other PPIDs.

CID Sequence

A CID sequence of the present invention can be any sequence that provides for interaction with the ER luminal protein chaperone such as BiP. By way of example, a CID sequence contains one or more of a proline rich segment. (e.g. 3 or 4 or 5 consecutive proline residues) optionally combined with one or more of valine, histidine, and leucine residues. By way of example, SEQ ID NO:3, derived from gamma-Zein, is useful as a CID sequence. While SEQ ID NO:3 comprises 8 imperfect repeats of a proline rich segment, a CID sequence can contain 1 or 2 or 3 or 4 or 5 or 6 or 7 or more such segments. In certain embodiments, the CID can comprise a variant of SEQ ID NO:3 that includes but is not limited to, conservatively substituted variants of SEQ ID NO:3, or protein domains with at least 80%, at least 85%, at least 90%, at least 95%, or at least 98% sequence identity to SEQ ID NO:3. In still other embodiments, the CID can be obtained from a rice prolamin coding region (Li et al. Science, 262:1054-1056, 1993; Saito et al. Journal of Experimental Botany, 60 (2): 615-627, 2009).

A CID can contain a hexapeptide PPPVHL (SEQ ID NO:18) in one or 2 or 3 or 4 or 5 or 6 or 7 or 8 or more perfect copies or imperfect copies.

Sites for Operable Insertion

A site for operable insertion can comprise any sequence that provides for operable insertion of the heterologous sequence in a construct of the instant invention. In certain embodiments, constructs provided herein can comprise one or more sequences encoding CID, TMD, and PPID domains where the site for operable insertion provides for an in-frame insertion of a sequence that encodes a polypeptide. In certain embodiments, the heterologous sequence comprising a site for operable insertion of a sequence that encodes a target protein comprises at least one restriction endonuclease recognition sequence. Restriction endonucleases and their recognition sequences are routinely used in the art to combine nucleic acid sequences to form recombinant nucleic acid constructs wherein joined sequences are operably linked. Further, it is understood that the restriction endonucleases and their recognition sequences disclosed herein are non-limiting examples and that other such restriction endonucleases and their recognition sequences not explicitly cited herein may be employed in the practice of the current invention. In still other embodiments, the site for operable insertion of the heterologous sequence can comprise a site for integration by homologous recombination. In still other embodiments, the site for operable insertion of the heterologous sequence can comprise a site-specific recombination recognition sequence. Examples of site-specific recombination recognition sequences include, but are not limited to, lox sites recognized by a bacteriophage P1 Cre recombinase, or FRT sites recognized by a yeast FLP recombinase. In still other embodiments, the site for operable insertion can comprise a Ligation Independent Cloning site that provides for DNA topoisomerase I mediated integration of the heterologous coding sequence. Various methods for operable insertion of heterologous sequences into specified sites are disclosed in U.S. Pat. No. 7,109,178, which is incorporated herein by reference with respect to its disclosure of Ligation Independent Cloning and directional cloning.

Target Protein Coding Sequence

A construct according to the present invention optionally comprises a sequence coding for a target protein. The target protein can be an otherwise be a naturally occurring protein other than the naturally occurring form of the protein from which the CID, TMD, PPID of the construct were obtained, a modified protein, or a non-naturally occurring protein. Typically, a target protein of the present invention codes for a portion of a protein or a complete protein. The protein can be one classified primarily as a structural protein or an enzymatically active protein. It can be used primarily as a nutritional protein or for some other physicochemical property (e.g. as a vaccine). It could be a completely novel protein with no known function, or it could be an artificially engineered protein.

It should be understood that the target protein coding sequence itself can also in certain embodiments contain one or more of a TMD, PPID, or CID domains. For example, if a target protein coding sequence contains a CID domain, this CID will function as a CID coding domain of the present invention. However, the composite encoded protein comprising the operably linked TMD, PPID, and/or CID domains of the construct and the TMD, PPID, and/or CID domains of the target protein would be distinct from the naturally occurring form of the protein from which the CID, TMD, and/or PPID of the construct were obtained.

A target protein can be any protein having therapeutic, nutraceutical, agricultural, or industrial uses.

A target protein can be heterologous to the recited eukaryotic host cells and is thus expressed in a second cell type that is different from the first-mentioned eukaryotic host cell.

The target protein can optionally exhibit at least 25 percent or 50 percent or 75 percent or at least 90 percent of the biological activity of the same polypeptide isolated from the above second cell type in an assay of the activity of that polypeptide.

In an optional embodiment, the target protein sequence is enhanced to encode a polypeptide having an increased content with a customized amino acid composition, e.g. one rich in essential amino acids, one deficient in amino acids that can be toxic to individuals with metabolic disorders (e.g. in the case of phenylketoneuria, deficient in phenylalanine), one rich in amino acids of otherwise limited availability to an individual animal, one rich in amino acids for individuals of special metabolic need (e.g. glutamine for athletes)

Optionally, the target protein sequence is host codon-optimized for a preferred pattern of codon usage. One skilled in the art can optimize codon usage based upon the host plant. For example, optimal codon usage in plants is described in U.S. Pat. No. 5,689,052.

Optionally, the target protein sequence is functionally linked to a sequence coding for a spacer amino acid sequence. The spacer amino acid sequence can be an amino acid sequence cleavable by enzymatic or chemical means or not cleavable.

An illustrative amino acid sequence is cleavable by a protease such as an enterokinase, Arg-C endoprotease, Glu-C endoprotease, Lys-C endoprotease, Factor Xa, SUMO proteases [Tauseef et al., 2005 Protein Expr. Purif. 2005 September 43(1):1-9] and the like. Alternatively, the spacer amino acid sequence corresponds to an auto-cleavable sequence such as the FMDV viral auto-processing 2A sequence, protein introns (inteins) such as the Ssp DNAb intein and the like as are commercially available from New England Biolabs and others. The use of an intein linker sequence is preferred as such sequences can be selectively induced to cause protein splicing and thereby eliminate themselves from an expressed, recovered, protein. Inteins are particularly interesting since they do not require large protein enzymes to reach their target site in order to cleave the target protein from the other expressed construct domains. This property may be particularly useful for direct isolation of proteins of interest from intact PB's. Alternatively, an amino acid sequence is encoded that is specifically cleavable by a chemical reagent, such as, for example, cyanogen bromide that cleaves at methionine residues.

In some embodiments, a target protein (e.g. fusion protein) produced by a host transformed by a present construct is in an insoluble form. Without being bound by theory, it is now believed that insolubility is due to interactions among the PPID peptide moiety mediated at least in part by the presence of cysteine residues. Moreover, the target protein is completed with eukaryotic chaperones and foldases derived from the ER and hence is held in a correctly folded conformation despite being tethered to an ER-derived membrane (and hence insoluble). The TMD-membrane and PPID-PPID interactions can be disrupted and the fusion protein solubilized by contacting the fusion protein with an aqueous buffer that contains a reducing agent and/or detergent. Reducing agents include, but are not limited to, agents such as dithiothreitol or 2-mercaptoethanol or β-mercaptoethanol. Conditions are chosen so as to not disrupt and unfold the attached biologically active protein of interest. The separated, solubilized fusion protein that contains the biologically active polypeptide is then collected or otherwise used. In addition, the two portions of the fusion can be cleaved from each other upon solubilization. It is to be understood that that cleavage need not be at the exact borders between the moieties.

In some embodiments, the solubilized fusion protein exhibits the biological activity of the biologically active polypeptide. In other embodiments, the fusion protein is dissolved or dispersed in a suitable buffer to exhibit the biological activity of the polypeptide.

In yet other embodiments, for steric interaction or size reasons the fusion protein has to be cleaved into its constituent parts before biological activity of the polypeptide is revealed. Thus, the biologically active polypeptide can be linked to the any of the CID, TMD, or PPID moieties by a by a spacer amino acid sequence that is cleavable by enzymatic or chemical means. Then, upon cleavage from the PB's, the target protein exhibits biological activity.

Hosts

Constructs of the present invention are useful for transforming a host. A host can be a non-human animal, fungus, plant, protozoa, algae, bacteria, or a yeast. The host can be isolated cells or tissue or organs or non-human organisms. When the host is an isolated cell or tissue or organs, the isolated cells, tissues, or organs can be from a human, a non-human animal, or plant. When the host is an isolated cell, the isolated cells can be a human cell, a non-human animal cell, a plant, a protozoan cell, an algal cell, a bacterial cell, or a yeast cell. The host can be a mature organism or an earlier developmental stage or undifferentiated tissue.

It should be understood that plant or animal host, as used herein, include, but are not limited to single cells, tissue, organs, organisms, explants, and the like

The skilled artisan can now recognize that the selection of a host (e.g. non-human animal, fungus, plant, protozoae, algae, bacteria, or a yeast) defines technical features of each of the domains of the present invention. For example, the selection or design of a CID depends upon the host. The same is equally true when other elements of the present invention are being selected or designed (e.g. promoter, TMD, PPID, terminator, and the like.).

Hosts include, but are not limited to, monocot plants, dicot plants, cells derived therefrom, and parts thereof, including roots, stems, leaves, flowers, tubers, and seeds.

Hosts also include, but are not limited to, eukaryotic cells that can be grown in suspension, in monolayer, as explants, or in aggregates.

ER Signal Sequence

Optionally, the heterologous polynucleotide of the present invention comprises an ER signal sequence. An ER signal sequence is any polynucleotide sequence that codes for an amino acid sequence known to result in the recognition of a given protein by the signal recognition particle on the endoplasmic reticulum resulting in the transport of the protein within the ER.

Illustrative signal peptides are the 19 residue gamma-zein signal peptide sequence shown in WO 2004003207 (US 20040005660), the 19 residue signal peptide sequence of alpha-gliadin or 21 residue gamma-gliadin signal peptide sequence (see, Altschuler et al., 1993 Plant Cell 5:443-450; Sugiyama et al., 1986 Plant Sci. 44:205-209; and Rafalski et al., 1984 EMBO J. 3(6):1409-11415 and the citations therein.) The pathogenesis-related protein of PR10 class includes a 25 residue signal peptide sequence that is also useful herein. Similarly functioning signal peptides from other plants and animals are also reported in the literature

Other examples of signal sequences which direct newly synthesized proteins to the endoplasmic reticulum in plant cells include barley lectin (Dombrowski J E, Schroeder M R, Bednarek S Y, Raikhel N V, 1993, Plant Cell 5:587-596), barley aleurain (Holwerda B C, Padgett H S, Rogers J C, 1992, Plant Cell 4:307-318), sweet potato sporamin (Matsuoka K, Nakamura K, 1991, Proc. Natl. Acad. Sci. USA 88:834-838), patatin (Sonnewald U, Braur M, von Schaewen A, Stitt M, Willmitzer L, 1991, Plant J. 1:95-106), soybean vegetative storage proteins (Mason H S, Guerrero F, Boyer J, Mullet J, 1988, Plant Mol. Biol. 11:845-856), and .beta.-fructosidase (Faye L, Chrispeels M J, 1989, Plant Physiol. 89:845-851).

While the skilled artisan can readily determine useful ER signal sequences, other examples include SEQ ID NO:4, SEQ ID NO:5, SEQ ID NO:6, and SEQ ID NO:7. Still other examples of ER signal sequences are described in Emanuelsson et al (J. Mol. Biol. 300, 1005-1016 (2000)).

UTR Translational Enhancer Domains

A construct of the present invention optionally comprises a 3′ UTR translational enhancer domain (“TED”). A TED can start at or near the stop codon and include contiguous nucleotides downstream (3′). A TED can be at least about 100 nucleotides or at least about 250 nucleotides or at least about 500 nucleotides. While the exact length is not critical to the invention, one skilled in the art can readily determine and optimize the length (e.g. by measuring and comparing translation levels). An exemplary and non-limiting 3′ UTR sequence is provided herein as SEQ ID NO:12.

Methods for Preparation

In optional embodiments, the target proteins are produced according to a method that comprises transforming a eukaryotic host cell system such as an animal, animal cell culture, plant or plant cell culture, fungus culture, insect cell culture or algae culture with a present construct further comprising a promoter and optional regulatory sequences present on either side of the nucleic acid sequences that encode the CID, TMD, CID, and target protein. Examples of such control sequences are well known and are present in commercially available vectors. The use of indirect means of introducing DNA, such as via viral transduction or infection, is also contemplated, and shall be used interchangeably with direct DNA delivery methods such as transfection.

The transformed host cell or entity can be maintained for a time period and under culture conditions suitable for expression of the fusion protein and assembly of the expressed fusion protein into PB's. Upon expression, the resulting fusion protein accumulates in the transformed host-system as PB's. The fusion protein can then be recovered from the host cells. The fusion protein can be isolated as part of the PB's or free from the PB's.

Culture conditions suitable for expression of the fusion protein are typically different for each type of host entity or host cell. However, those conditions are known by skilled workers and are readily determined. Similarly, the duration of maintenance can differ with the host cells and with the amount of fusion protein desired to be prepared. Again, those conditions are well known and can readily be determined in specific situations. Additionally, specific culture conditions can be obtained from the citations herein.

A DNA segment that encodes proteins of present invention can be synthesized by chemical techniques, for example, the phosphotriester method of Matteucci et al., 1981 J. Am. Chem. Soc., 103:3185. Of course, by chemically synthesizing the coding sequence, any desired modifications can be made simply by substituting the appropriate bases for those encoding the native amino acid residue sequence. However, DNA segments including sequences specifically discussed herein are preferred.

DNA segments containing a gene encoding the fusion protein are preferably obtained from recombinant DNA molecules (plasmid vectors) containing that gene. A vector that directs the expression of a fusion protein gene in a host cell is referred to herein as an “expression vector”.

An expression vector contains expression control elements including the promoter. The fusion protein-coding gene is operatively linked to the expression vector to permit the promoter sequence to direct RNA polymerase binding and expression of the fusion protein-encoding gene. Useful in expressing the polypeptide coding gene are promoters that are inducible, viral, synthetic, constitutive as described by Paszkowski et al., 1989 EMBO J., 3:2719 and Odell et al., 1985 Nature, 313:810, as well as temporally regulated, spatially regulated, and spatiotemporally regulated as given in Chua et al., 1989 Science, 244:174-181.

Expression vectors compatible with eukaryotic cells, such as those compatible with cells of mammals, algae or insects and the like, are contemplated herein. Such expression vectors can also be used to form the recombinant DNA molecules of the present invention. Eukaryotic cell expression vectors are well known in the art and are available from several commercial sources. Normally, such vectors contain one or more convenient restriction sites for insertion of the desired DNA segment and promoter sequences. Optionally, such vectors contain a selectable marker specific for use in eukaryotic cells.

Production of a fusion protein by recombinant DNA expression in mammalian cells can be accomplished by using a recombinant DNA vector that expresses the fusion protein gene in Chinese hamster ovary (CHO) host cells, Cos1 monkey host and human 293T host cells. This is accomplished using procedures that are well known in the art and are described in more detail in Sambrook et al., Molecular Cloning: A Laboratory Manual, 2.sup.nd ed., Cold Spring Harbor Laboratories (1989).

An insect cell system can also be used to express a contemplated fusion protein. For example, in one such system Autographa californica nuclear polyhedrosis virus (AcNPV) or baculovirus is used as a vector to express foreign genes in Spodoptera frugiperda cells or in Trichoplusia larvae. The sequences encoding a fusion protein can be cloned into a non-essential region of the virus, such as the polyhedrin gene, and placed under control of the polyhedrin promoter. Successful insertion of a fusion protein sequence renders the polyhedrin gene inactive and produces recombinant virus lacking coat protein. The recombinant viruses can then be used to infect, for example, S. frugiperda cells or Trichoplusia larvae in which the fusion protein can be expressed, for example as described in Engelhard et al. (1994) Proc. Natl. Acad. Sci., USA, 91:3224-3227; and V. Luckow, “Insect Cell Expression Technology”, pages 183-218, in Protein Engineering: Principles and Practice, J. L. Cleland et al. eds., Wiley-Liss, Inc, 1996). Heterologous genes placed under the control of the □olyhedron promoter of the Autographa californica nuclear polyhedrosis virus (AcNPV) are often expressed at high levels during the late stages of infection.

Recombinant baculoviruses containing the fusion protein gene are constructed using the baculovirus shuttle vector system (Luckow et al., 1993 J. Virol., 67:4566-4579], sold commercially as the Bac-To-Bac.™. baculovirus expression system (Life Technologies, Carlsbad, Calif., USA). Stocks of recombinant viruses are prepared and expression of the recombinant protein is monitored by standard protocols (O'Reilly et al., Baculovirus Expression Vectors: A Laboratory Manual, W.H. Freeman and Company, New York, 1992; and King et al., The Baculovirus Expression System: A Laboratory Guide, Chapman & Hall, London, 1992). Use of baculovirus or other delivery vectors in mammalian cells, such as the ‘BacMam’ system described by T. Kost and coworkers (see, for example Merrihew et al., 2004 Methods Mol. Biol. 246:355-365), or other such systems as are known to those skilled in the art are also contemplated in the instant invention.

The choice of which expression vector and ultimately to which promoter a fusion protein-encoding gene is operatively linked depends directly on the functional properties desired, e.g., the location and timing of protein expression, and the host cell to be transformed. These are well known limitations inherent in the art of constructing recombinant DNA molecules. However, a vector useful in practicing the present invention can direct the replication, and preferably also the expression (for an expression vector) of the fusion protein gene included in the DNA segment to which it is operatively linked.

Typical vectors useful for expression of genes in cells from higher plants and mammals are well known in the art and include plant vectors derived from the tumor-inducing (Ti) plasmid of Agrobacterium tumefaciens described by Rogers et al. (1987) Meth. In Enzymol., 153:253-277 and mammalian expression vectors pKSV-10, above, and pCI-neo (Promega Corp., #E1841, Madison, Wis.). However, several other expression vector systems are known to function in plants including pCaMVCN transfer control vector described by Fromm et al. ((1985) Proc. Natl. Acad. Sci. USA, 82:58-24). Plasmid pCaMVCN (available from Pharmacia, Piscataway, N.J.) includes the cauliflower mosaic virus CaMV 35S promoter.

Transfection of plant cells using Agrobacterium tumefaciens is typically best carried out on dicotyledonous plants. Monocots are usually most readily transformed by so-called direct gene transfer of protoplasts. Direct gene transfer is usually carried out by electroporation, by polyethyleneglycol-mediated transfer or bombardment of cells by microprojectiles carrying the needed DNA. These methods of transfection are well-known in the art and need not be further discussed herein. Methods of regenerating whole plants from transfected cells and protoplasts are also well-known, as are techniques for obtaining a desired protein from plant tissues. See, also, U.S. Pat. Nos. 5,618,988 and 5,679,880 and the citations therein.

A transgenic plant formed using Agrobacterium transformation, electroporation or other methods typically contains a single gene on one chromosome. Such transgenic plants can be referred to as being heterozygous for the added gene. However, inasmuch as use of the word “heterozygous” usually implies the presence of a complementary gene at the same locus of the second chromosome of a pair of chromosomes, and there is no such gene in a plant containing one added gene as here, it is believed that a more accurate name for such a plant is an independent segregant, because the added, exogenous chimer molecule-encoding gene segregates independently during mitosis and meiosis. A transgenic plant containing an organ-enhanced promoter driving a single structural gene that encodes a contemplated HBc chimeric molecule; i.e., an independent segregant, is a preferred transgenic plant.

More preferred is a transgenic plant that is homozygous for the added structural gene; i.e., a transgenic plant that contains two added genes, one gene at the same locus on each chromosome of a chromosome pair. A homozygous transgenic plant can be obtained by sexually mating (selfing) an independent segregant transgenic plant that contains a single added gene, germinating some of the seed produced and analyzing the resulting plants produced for enhanced chimer particle accumulation relative to a control (native, non-transgenic) or an independent segregant transgenic plant. A homozygous transgenic plant exhibits enhanced chimer particle accumulation as compared to both a native, non-transgenic plant and an independent segregant transgenic plant.

It is to be understood that two different transgenic plants can also be mated to produce offspring that contain two independently segregating added, exogenous (heterologous) genes. Selfing of appropriate progeny can produce plants that are homozygous for both added, exogenous genes that encode a chimeric HBc molecule. Back-crossing to a parental plant and out-crossing with a non-transgenic plant are also contemplated.

Methods of Purifying Target Proteins

Target proteins produced according to the present invention localized to regions of different density are formed in homogenates to provide a region that contains a relatively enhanced concentration of the PB's and a region that contains a relatively depleted concentration of the PB's. The PB-depleted region is separated from the region of relatively enhanced concentration of PB's, thereby purifying the target protein. The region of relatively enhanced concentration of PB's can thereafter be collected or can be treated with one or more reagents or subjected to one or more procedures prior to isolation of the PB's or the target protein therein. In some embodiments, the collected PB's are used as is, without the need to isolate the target protein, as where the PB's are used as an oral vaccine. The target protein containing the biologically active polypeptide can be obtained from the collected PB's by dissolution of the surrounding membrane in an aqueous buffer containing a detergent and a reducing agent as discussed previously. Illustrative reducing agents include, but are not limited to, 2-mercaptoethanol, thioglycolic acid and thioglycolate salts, dithiothreitol (DTT), sulfite or bisulfite ions, followed by usual protein isolation methods. Sodium dodecyl sulfate (SDS) is the preferred detergent, although other ionic (deoxycholate, 'N-Lauroylsarcosine, and the like), non-ionic (Tween.™. 20, Nonidet.™. P-40, octyl glucoside and the like) and zwitterionic (CHAPS, Zwittergent.™. 3-X serie and the like) surfactants can be used. A minimal amount of surfactant that dissolves or disperses the fusion protein can be utilized.

The present invention provides an easy purification process of the PB from transformed tissues by methods including, but not limited to, gradient centrifugation. Simply, the crude protein extract is placed as a layer on a 5%-15% sucrose or ficoll cushion and briefly centrifuged. The PB precipitate and can be collected from the bottom of the centrifuge tube following centrifugation.

Unexpected Technical Features of the Present Invention

Surprisingly, the present invention results in a plant with superior features. For example, the invention allows for the production of a target protein of nearly any type in nearly any host. It can be produced as a protein that has desirable physicochemical properties (such as solubility or stability under various conditions); it can be produced as a fusion protein linked to residues that aid purification or identification, or enhance its utility.

The present invention is especially useful for producing proteins that otherwise are sensitive to degradation and, through sequestration by compartmentalization in PB's or by dense packing of the protein target taught herein, can demonstrate remarkable stability.

The present invention is especially useful for producing proteins that are non-glycosylated, due to optionally providing for escape from the Golgi apparatus.

The present invention is especially useful for producing proteins that require low humidity to preserve function (for example, such proteins can be targeted to ER-derived vesicles in a seed and stored for months or years and preserve nutritional value, enzymatic activity, or a desired property.

The present invention is also useful in that it can provide for facile purification of target proteins as PB.

The present invention is especially useful for producing proteins in a tissue specific or tissue non-specific manner by promoter selection.

The constructs of the present invention can be combined with other useful features, such as additional regulatory elements that allow the gene to be turned on or off by temporal or external signals.

### EXAMPLES

### Example 1

### Production in Transformed Plants

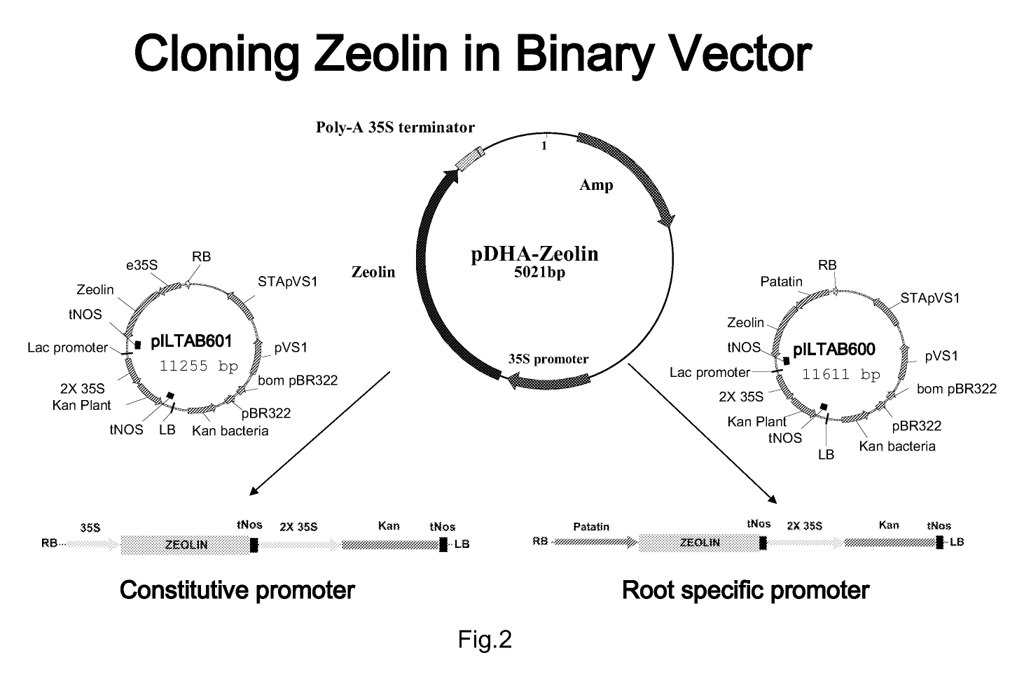

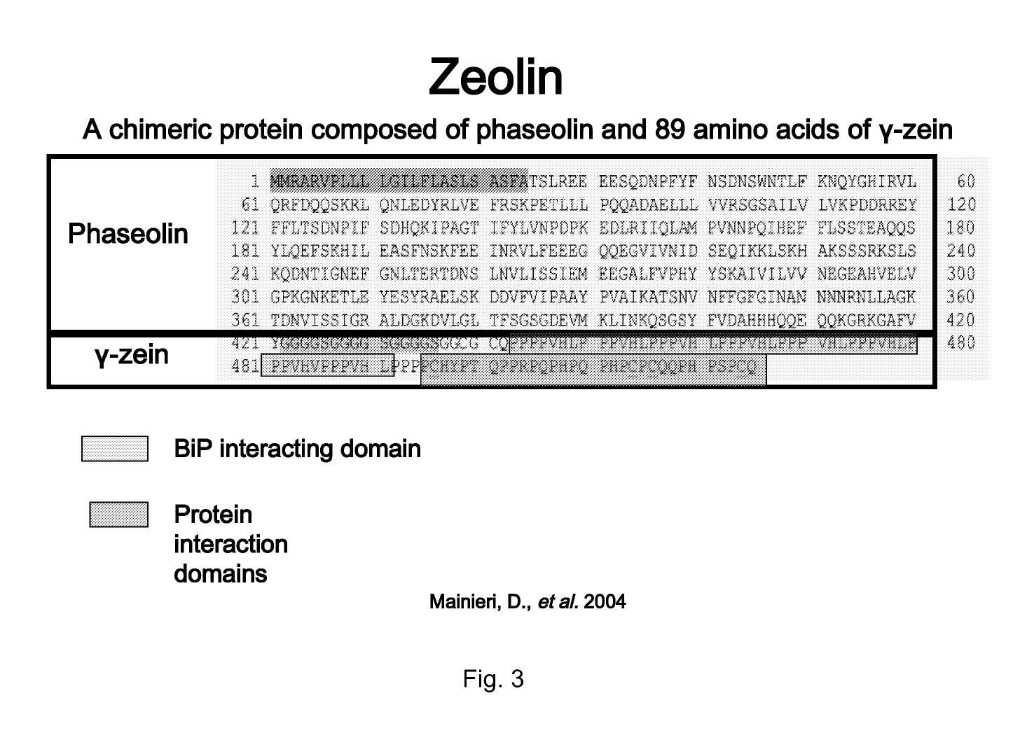

A construct for transforming a plant to produce Zeolin was synthesized as set forth in FIG. 2. Plants transformed by this construct produced Zeolin of the sequence set forth in FIG. 3. Zeolin does not contain a TMD of the present invention.

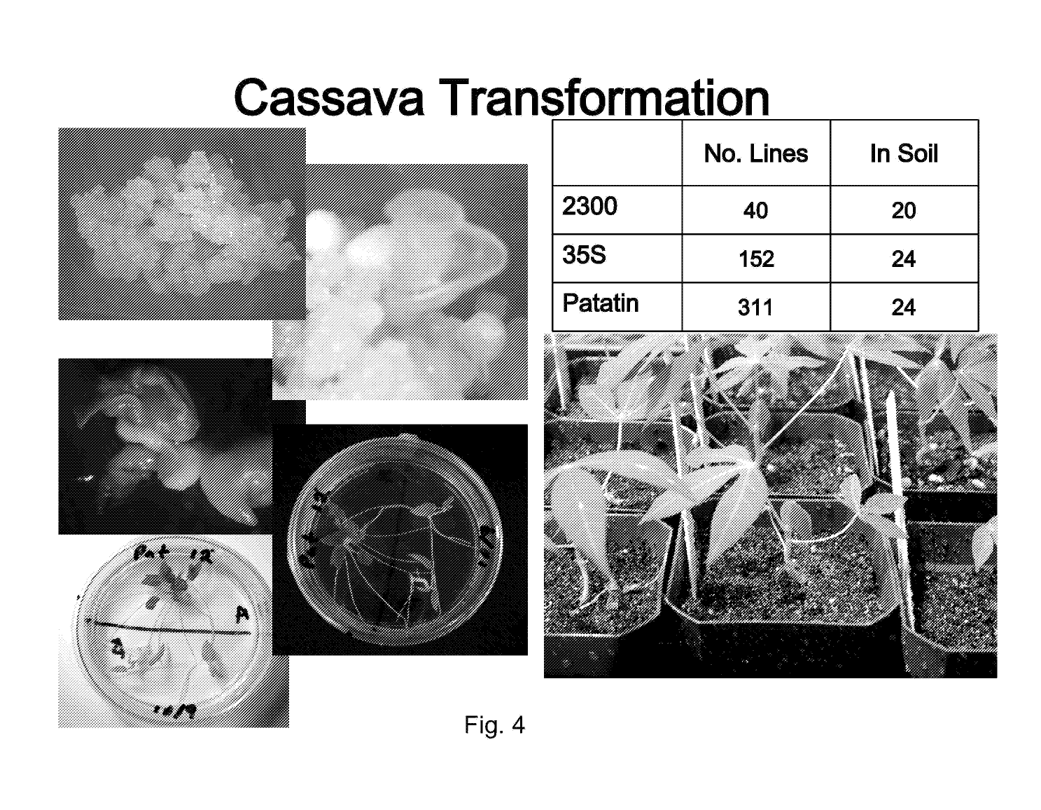

Cassava was transformed with the construct of Example 1 and independent isolates were cultivated as shown in FIG. 4. The amino acid sequence of Zeolin is provided as SEQ ID NO: 13 and the DNA sequence encoding Zeolin is provided as SEQ ID NO: 17.

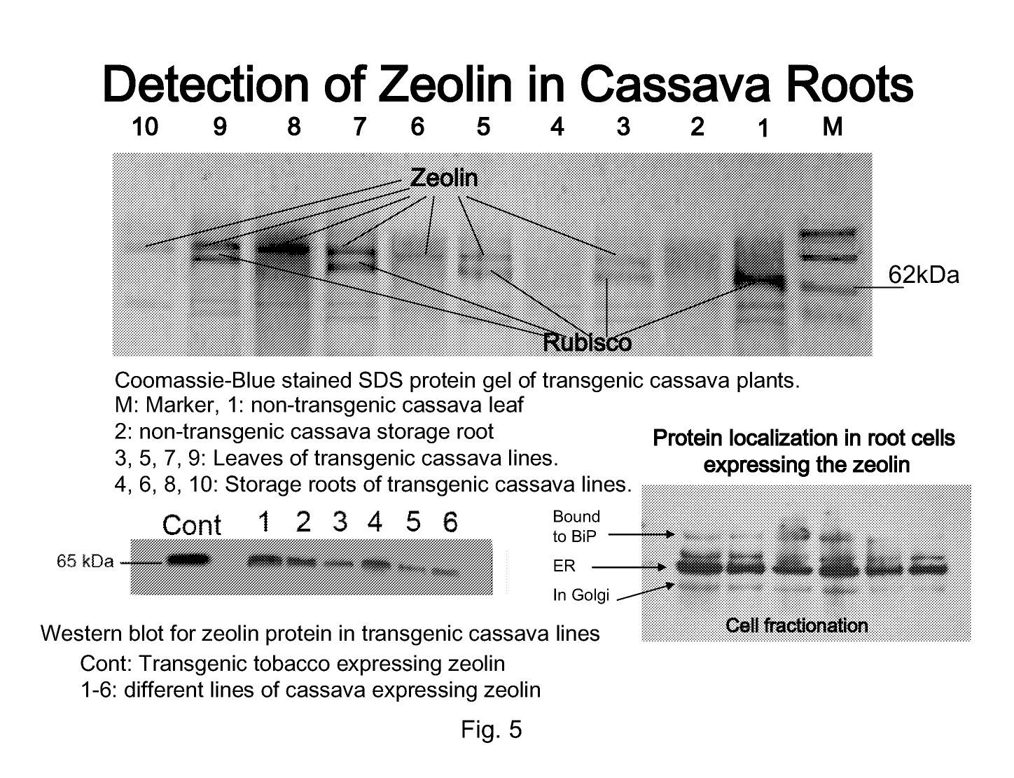

Zeolin production was examined by SDS-PAGE and Western analysis in Cassava root as shown in FIG. 5.

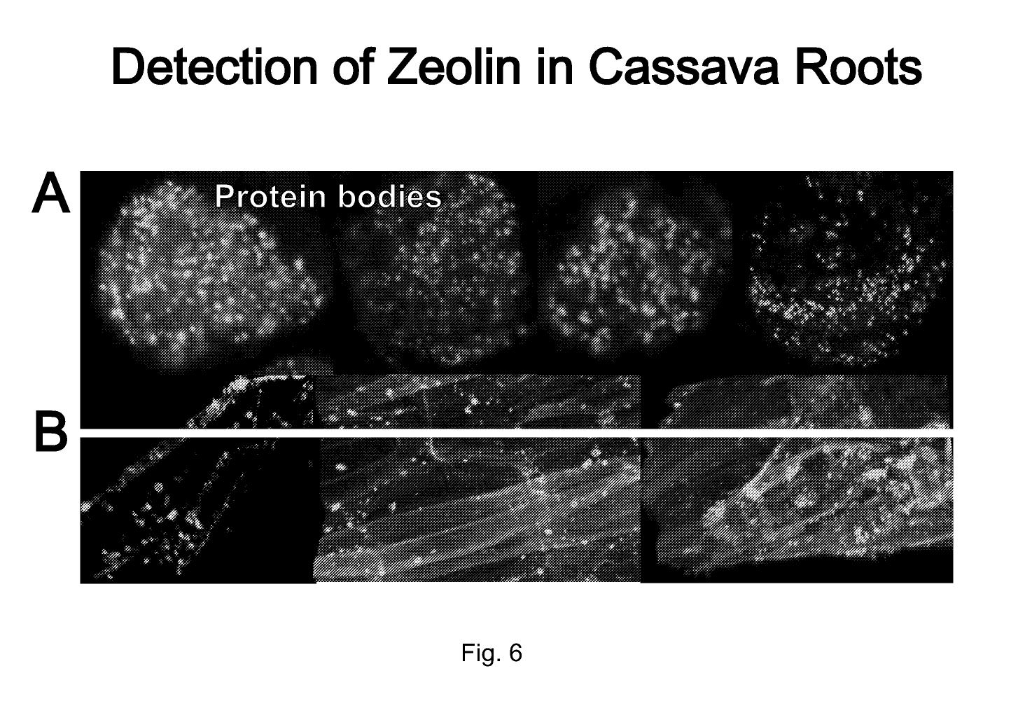

Intracellular localization of Zeolin was examined in Cassava root. Cells were fixed, hybridized with specific Zeolin antibodies, labeled with fluorescent secondary antibodies and imaged at an exposure of 100 ms. As shown in FIG. 6, confocal immunomicroscopy showed that Zeolin was present as punctuate florescence indicating spherical protein bodies localized in the ER of transgenic cells. A: Tobacco BY2 cells and B: Cassava root cells. The fluorescent

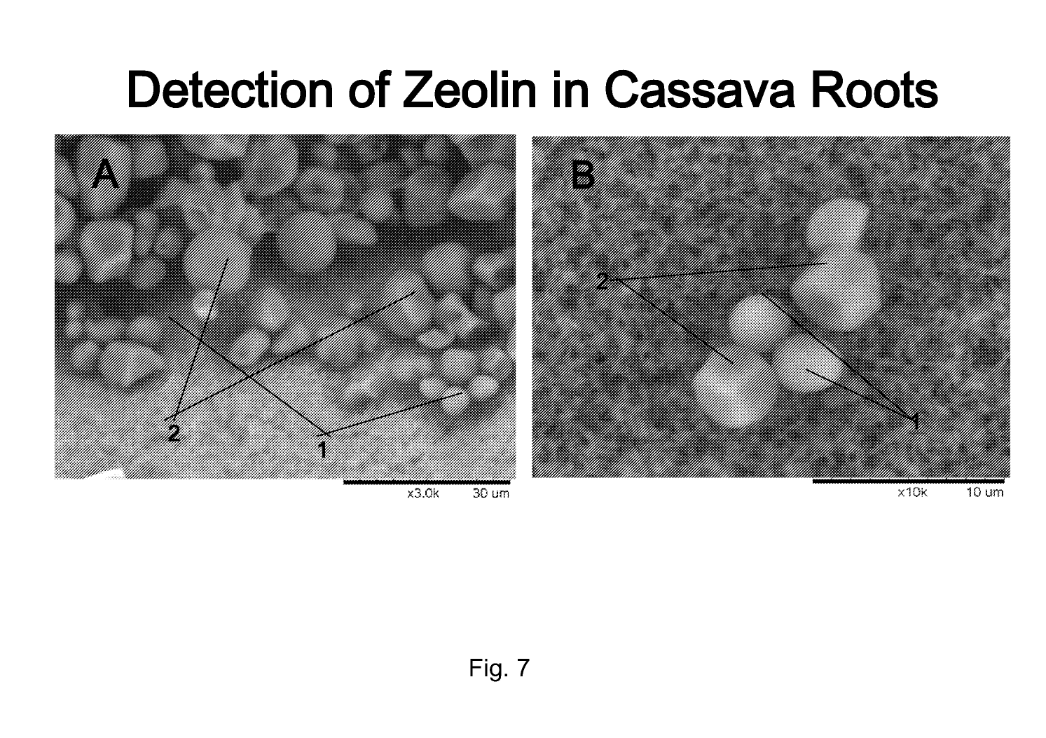

Purified protein bodies were examined by SEM as shown in FIG. 7. (A) BY2 cells and (B) Cassava storage parenchyma root cells. The “1” indicates PB; “2” indicates vacuolar bodies. The SEM images show different sizes of PB recovered from the ER and vacuole of transgenic cells. For confirmation, the purified extracts were hybridized with specific Zeolin antibodies, labeled with a fluorescent secondary antibodies, and counted under the confocal microscope. While 58% of PB in transgenic BY2 cells showed presence of Zeolin, 91% of such bodies in cassava storage parenchyma root cells were positive for the transgenic storage protein.

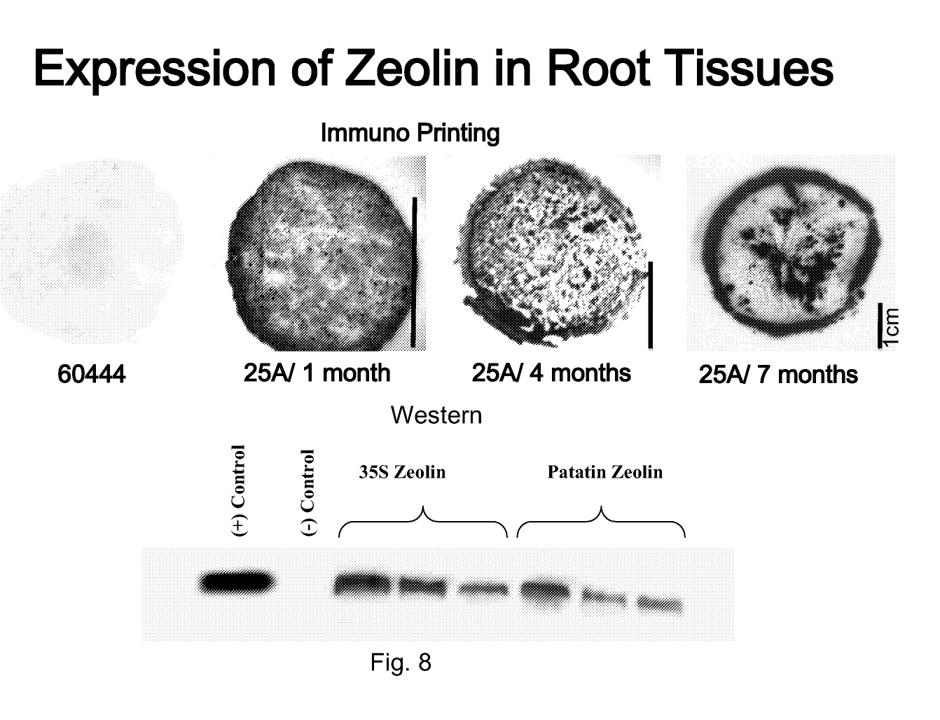

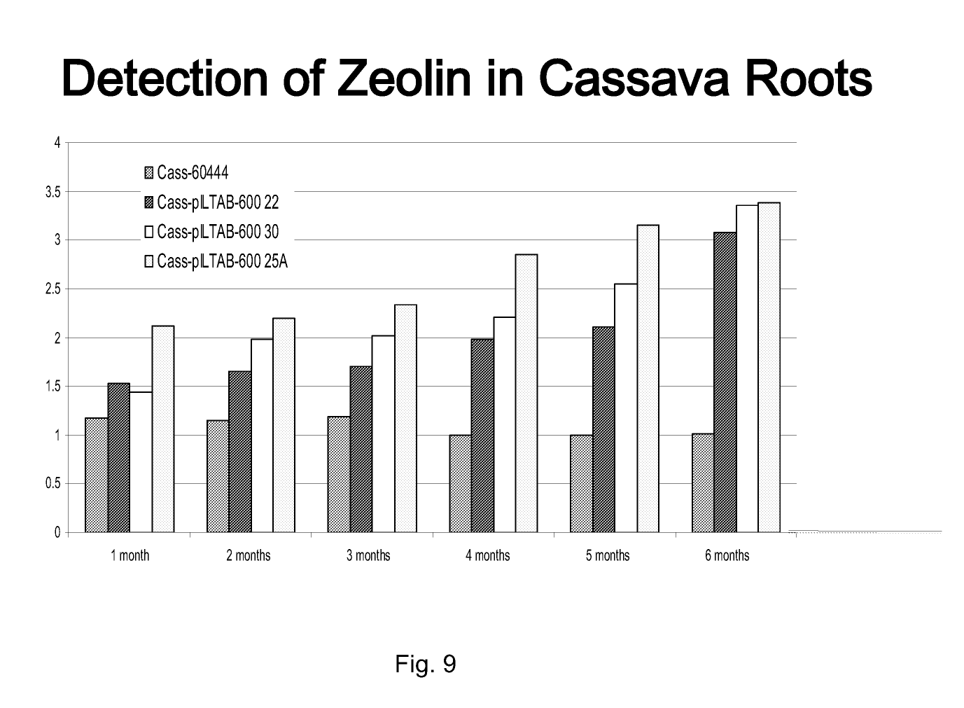

The temporal expression of Zeolin in cassava root was examined as shown in FIG. 8 and FIG. 9. Protein content in transgenic lines of cassava storage roots expressing Zeolin were examined at the age of one to six months old plants. The protein content accumulates with time compared with the non-transgenic control 60444, total protein is extracted from fresh tissue and measured by the CB-X protein assay (G-Biosciences, St. Louis, Mo. 63043. USA).

### Example 2

### Production of Cassettes

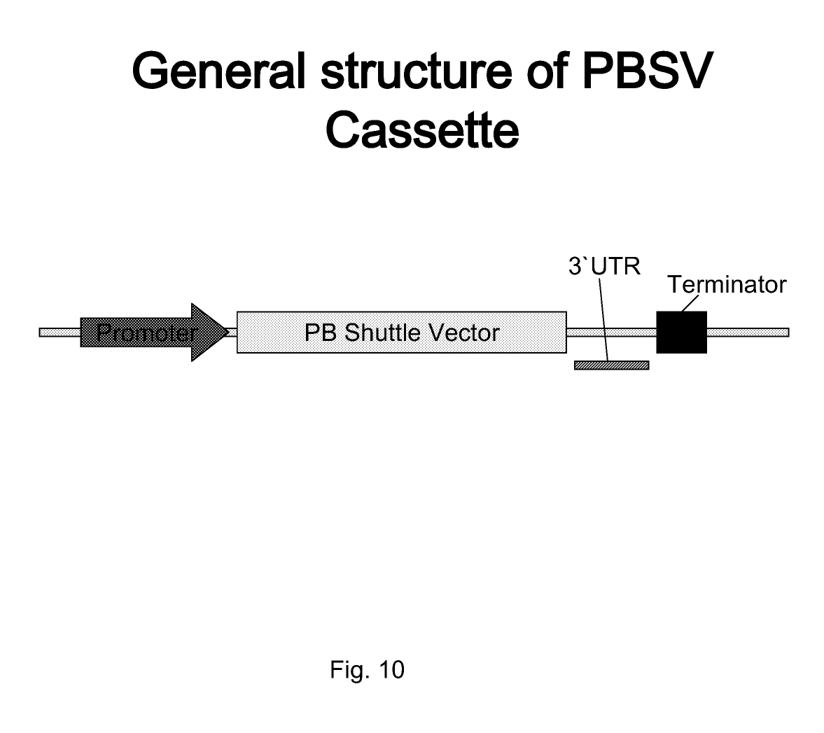

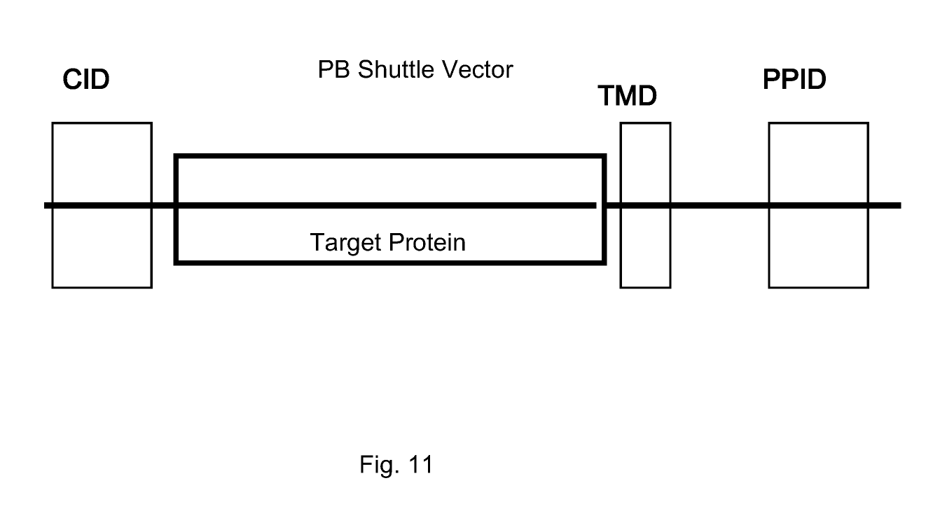

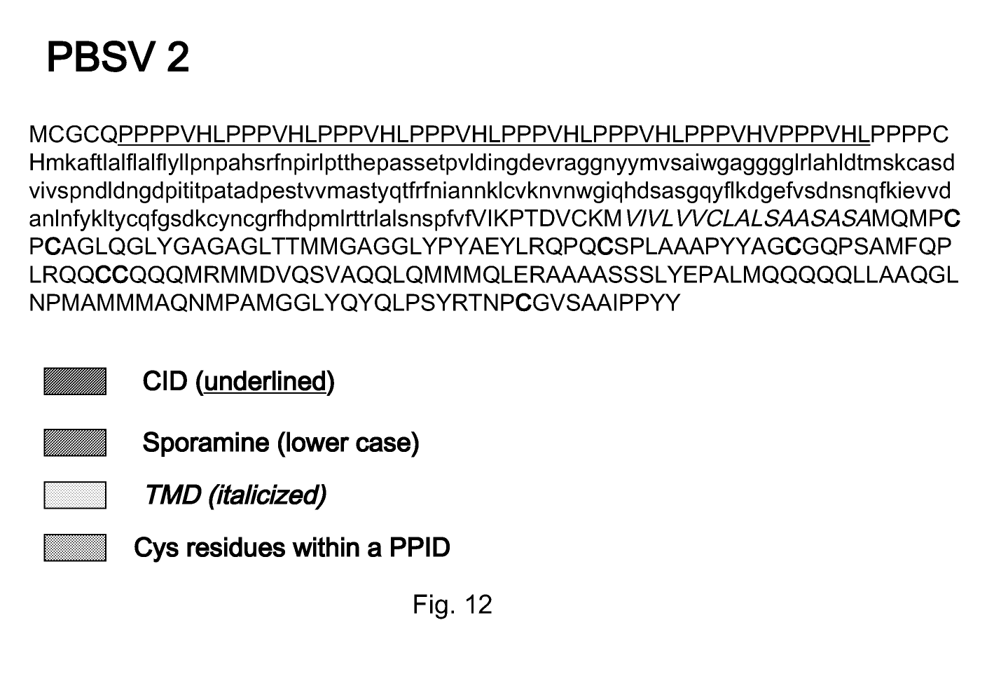

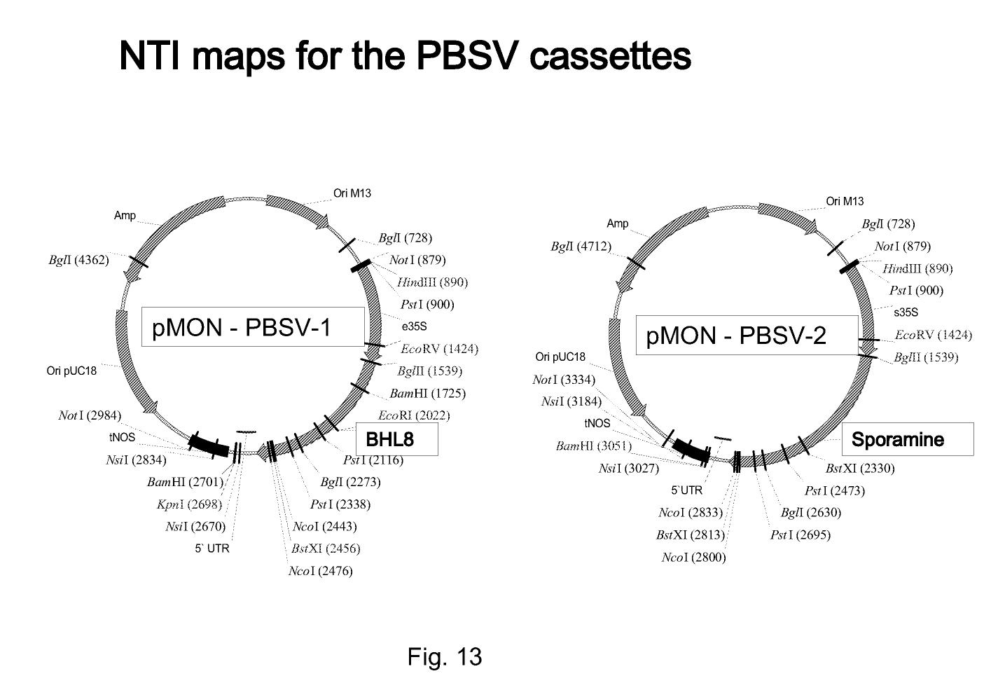

Constructs of the present invention were produced as cassettes to transform different target proteins in a host plant such that the target protein was shuttled to protein bodies. FIG. 10 shows the general structure of the cassette. FIG. 11 shows the basic structure of the constructs (the “PB shuttle vector”). FIG. 12 shows the amino acid sequence for PBSV-2 (SEQ ID NO:9). FIG. 13 shows the NTI maps for the PBSV cassettes. The target protein for PBSV-1 (SEQ ID NO:8), PBSV-2 (SEQ ID NO:9), and PBSV-3 (SEQ ID NO:10) are BHL8, Sporamine, and GFP, respectively. BHL8 is a mutated form of the Barley High Lysine protein that lacks chymotrypsin inhibitory activity (Roesler and Rao, J. Agric. Food Chem. 2001, 49, 3443-3451). The coding sequence for PBSV-1 is provided as SEQ ID NO:14, the coding sequence for PBSV-2 is provided as SEQ ID NO:21, and the coding sequence for PBSV-3 is provided as SEQ ID NO:15.

### Example 3

### Transformation of BY2 Cells with Present Constructs and Comparator Constructs

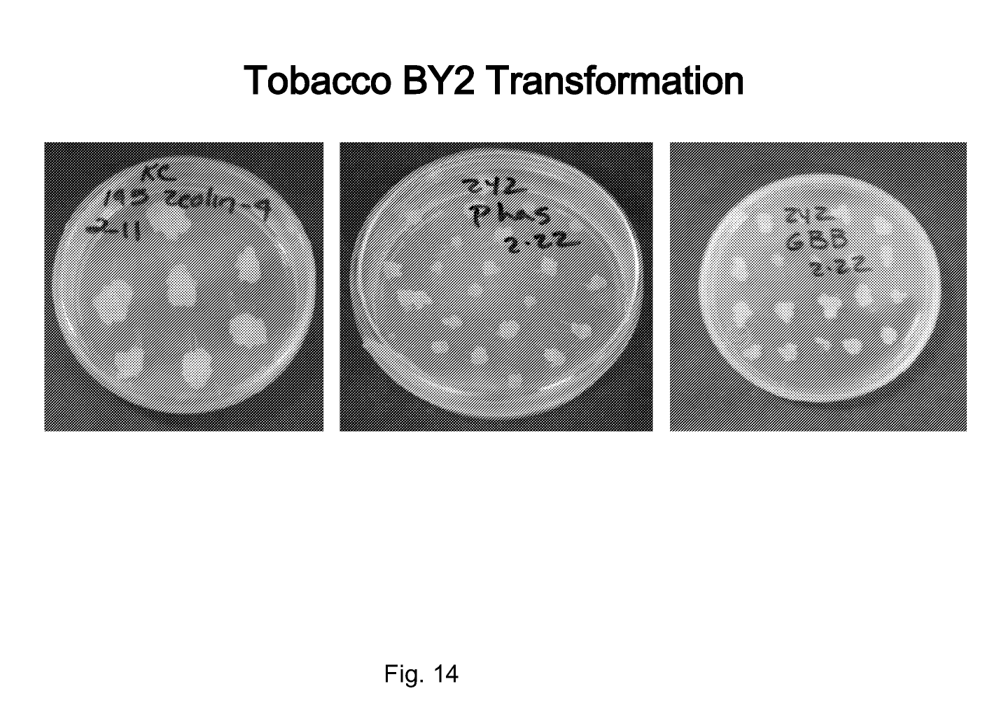

Constructs set forth in Table 1 and PBSV-1 were made and used to transform BY2 cells using Agrobacterium mediated transformation. Briefly, constructs were cloned in pCambia 2300 based binary vectors and transformed into Agrobacterium tumefaciens LBA4404. Agrobacterium with the binary vector were allowed to infect BY2 cell suspension and incubated until T-DNA (Constructs) were transferred into BY2 cells. BY2 cells were grown on nutritional media to grow and form undifferentiated calli. BY2 calli were maintained in petri-dishes with media for analysis (see FIG. 14).

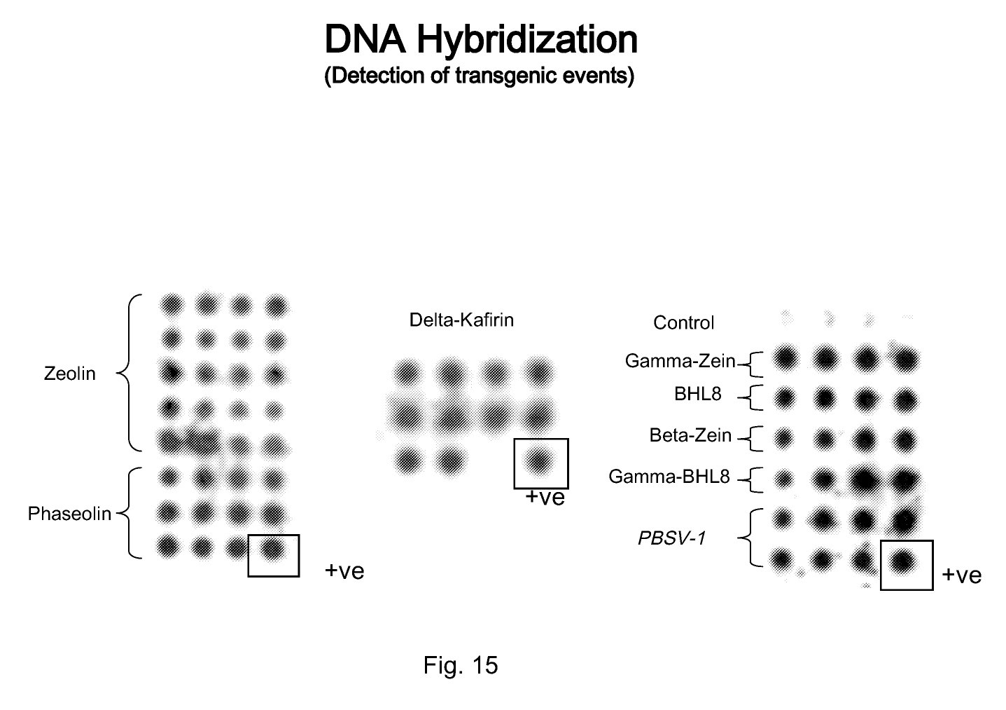

Transformed BY2 cell lines were identified by DNA hybridization. DNA was extracted from grown BY2 calli cells by grinding in Dellaporta extraction buffer. Extracted DNA was blotted on Nylon membrane and hybridized with specific probes against cloned gene constructs. Exemplary results are shown in FIG. 15.

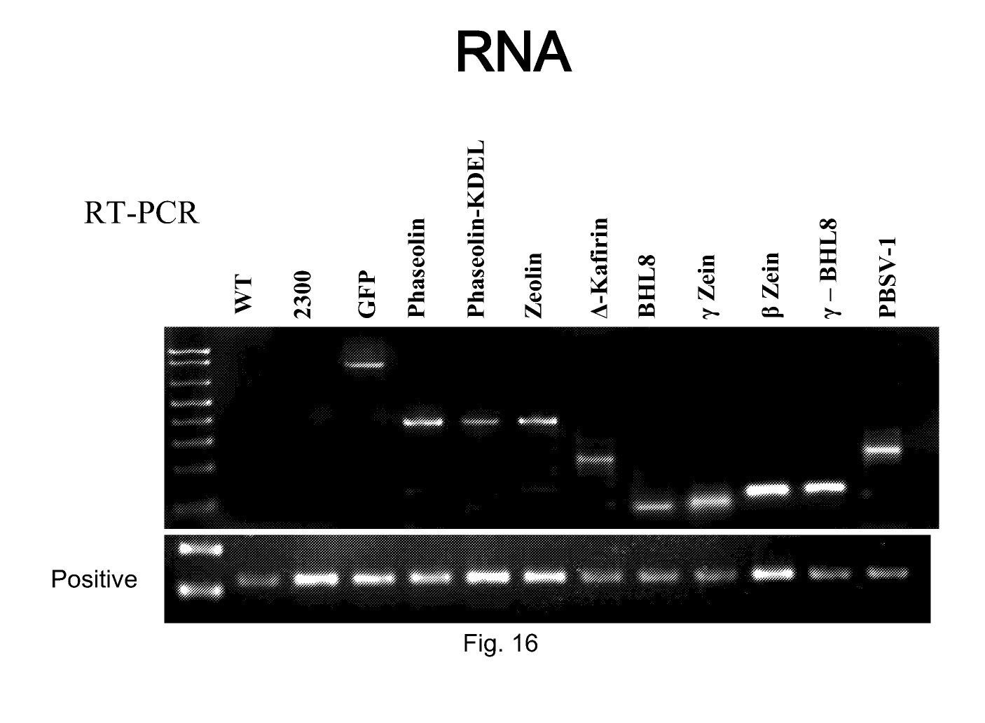

Transcription of constructs in transgenic BY2 cells was examined. RNA was extracted from the transformed BY2 cell lines. Reverse transcription was performed on the extracted RNA to make complementary DNA. PCR was made with specific primer sets for each construct. Exemplary results are shown in FIG. 16 where transcript level was determined by comparing to positive RNA transcript in BY2 cell lines. Hosts transformed with instant constructs (e.g. PBSV-1) demonstrate substantial mRNA production in host cells (in the range of or greater than the other comparator constructs.

TABLE 1
Test Constructs

| | Cyto- | Protein | MW of the | |
|---|---|---|---|---|
| | plasm | Bodies | target protein | |
| Phaseolin | + | − | 50.5 | kDa |
| Phaseolin-KDEL | − | + | 65 | kDa |
| Zeoline | − | + | 18.9 | kDa |
| δ-Kafirine | + | − | 10.9 | kDa |
| γ-Zein | − | + | 20.6 | kDa |
| β-Zein | − | + | 21.7 | kDa |
| BHL8 | − | + | 27 | kDa |
| γ-BHL8 | − | + | 36.3 | kDa |
| PBSV-1 | | + | 49.6 | kDa |
| PBSV-2 | − | + | 21.7 | kDa |

### Example 4

### Production of the Target Protein in BY2 Cells Transformed with Instant Constructs

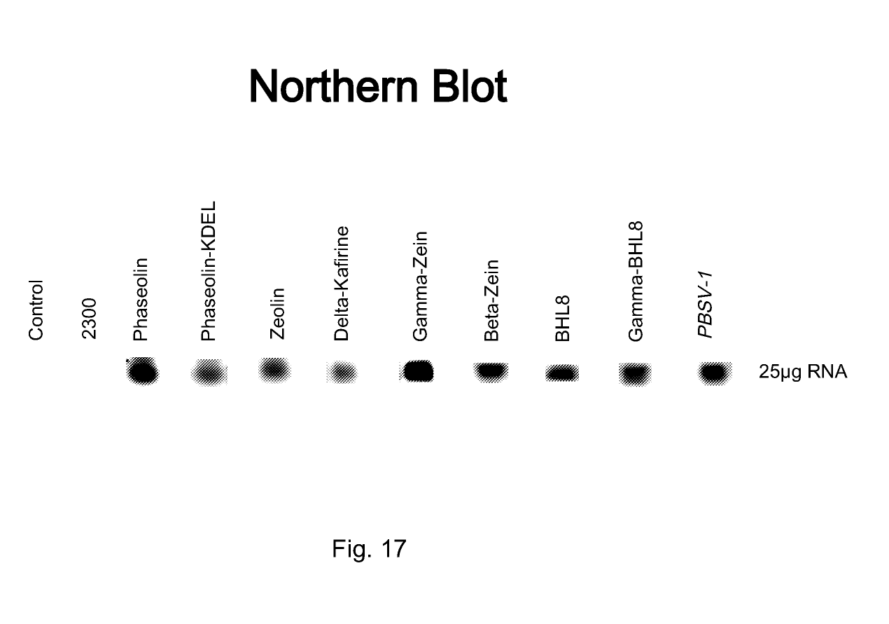

The BY2 cells transformed with the constructs from the example above (and set forth in Table 1) were analyzed by Northern analysis. Extracted RNA was loaded on an RNA gel and electrophoresed. The RNA was transferred on to nylon membrane and hybridized with specific probes against gene constructs. Exemplary results are shown in FIG. 17, demonstrating the utility of the present invention to drive the production of an RNA species of interest (i.e. for the target protein).

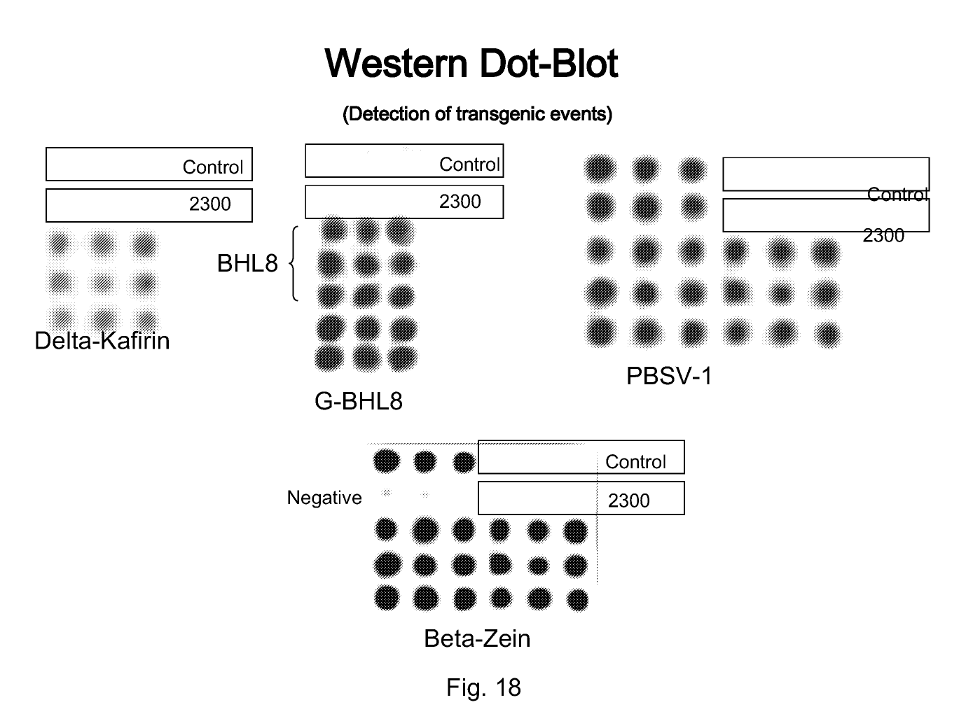

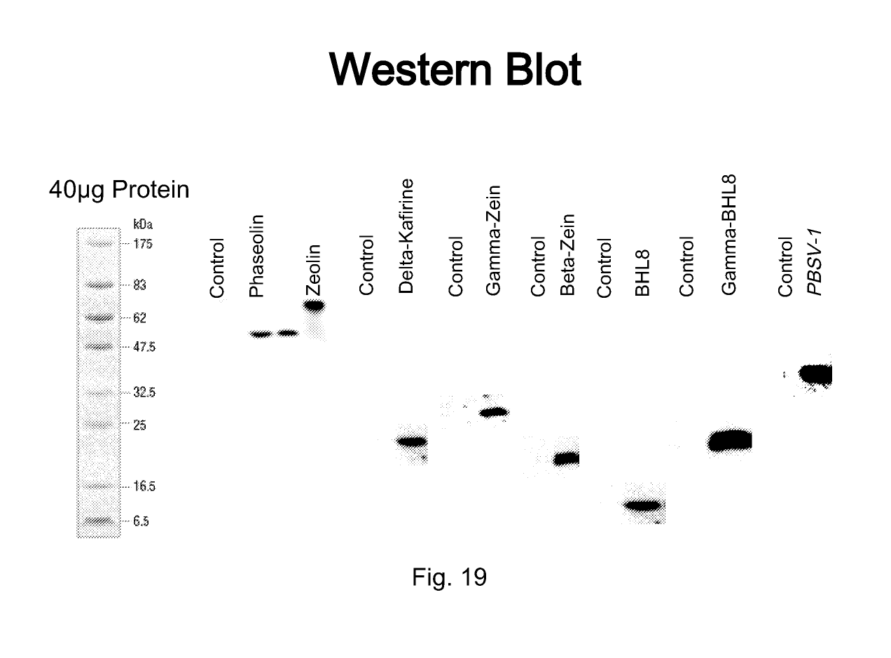

Protein production from instant constructs expressed in transgenic BY2 cell lines was examined. Protein was extracted from transgenic BY2 cell lines showing positive DNA & RNA by grinding in protein extraction buffer. Protein extract was first blotted on nitrocellulose membrane to detect protein translation in the cell by dot-blot western analysis. Exemplary results are shown in FIG. 18. Hosts transformed with instant constructs (e.g. PBSV-1) demonstrate substantial protein productions (in the range of or greater than the other comparator constructs). Thus, present constructs provide a mechanism for achieving a superior quantity of a target protein.

Extracted proteins were also loaded on SDS-PAGE and transferred onto nitrocellulose membrane for western blotting. Exemplary results are shown in FIG. 19.

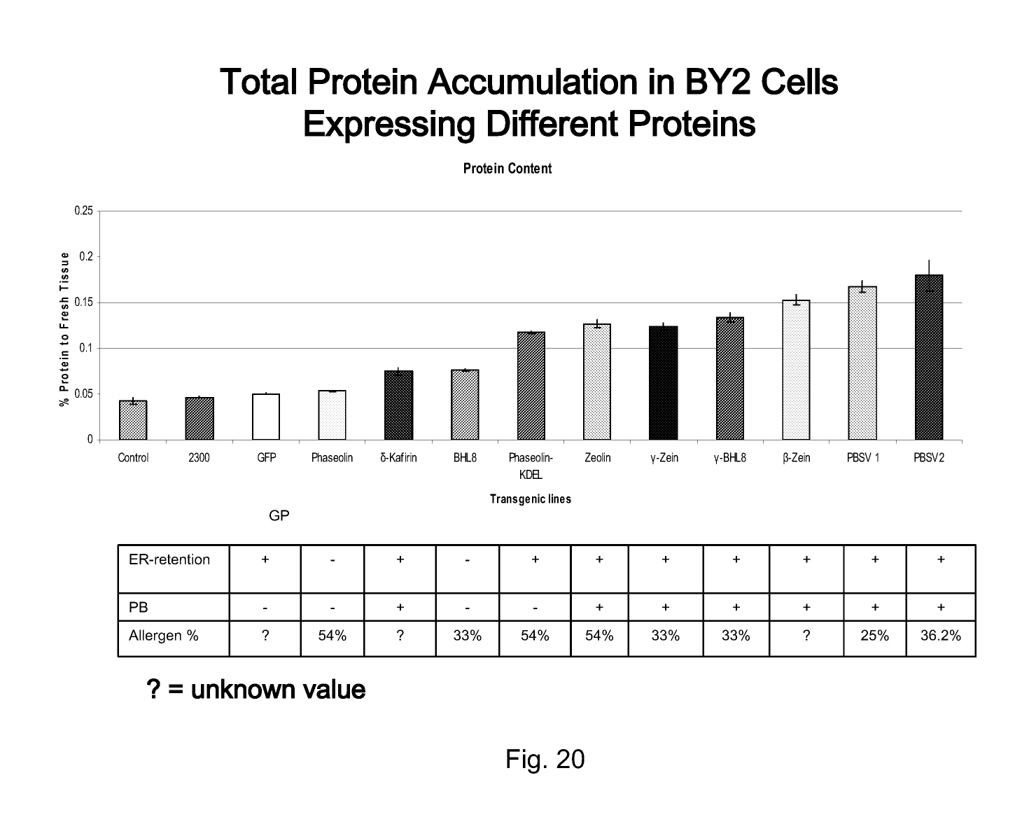

Total target protein accumulation in BY2 cells transformed with instant constructs was quantified. Total protein extract was subjected to quantity determination through CB-X protein analysis kit to measure protein amount. Protein extracts were measured in spectrophotometer and determined. As shown in FIG. 20, target proteins transformed with PBSV-1 or PBSV-2 are able to accumulate target protein to levels that exceed comparator constructs lacking one or more of the technical features of the present invention.

### Example 5

### Microscopy of Production of the Target Protein in Various Constructs of the Present Invention in Transformed Plants

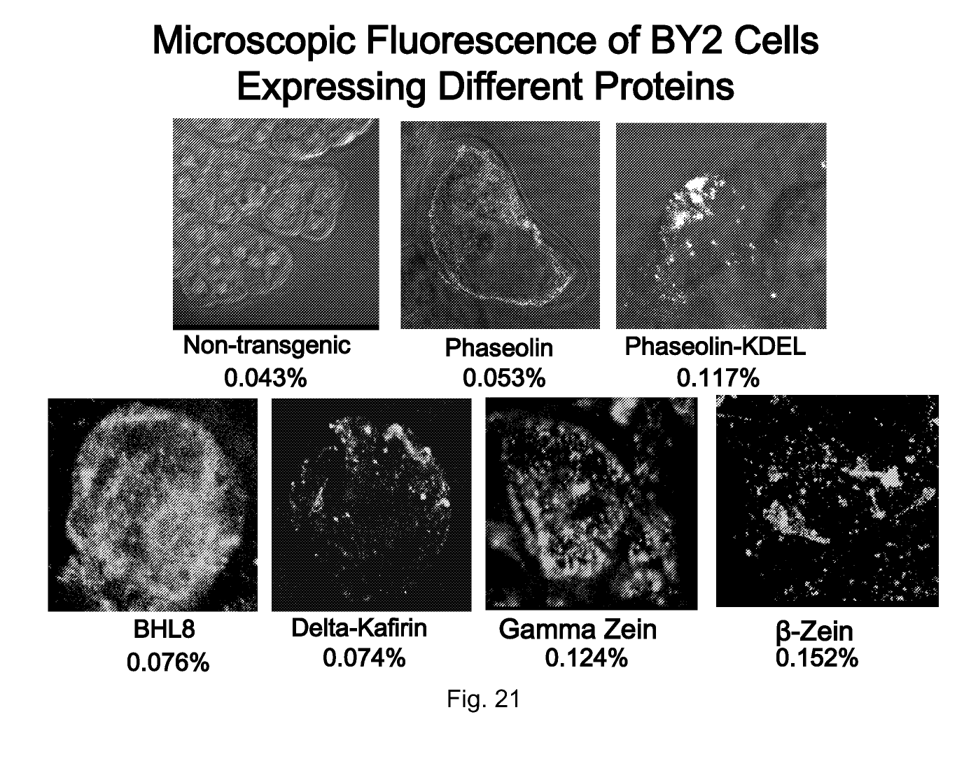

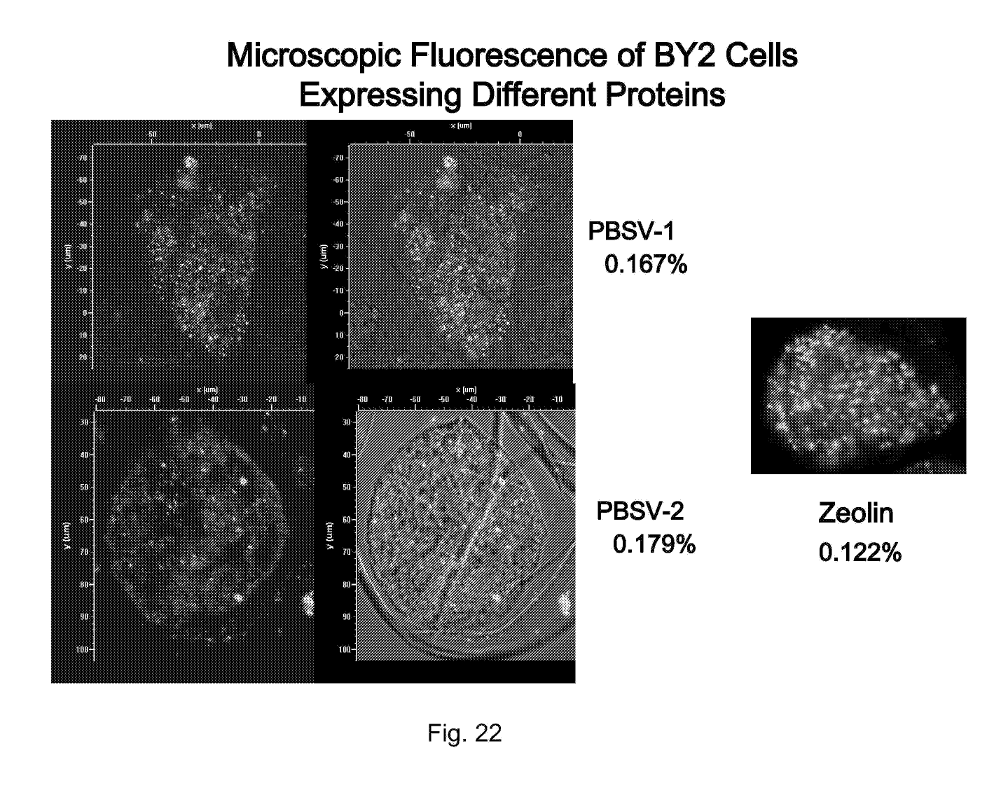

Subcellular localization of accumulated target protein produced in BY2 cells transformed with PBSV-1 and PBSV-2 was examined in comparison to the other constructs set forth in the previous examples. Transgenic BY2 cells were fixed in para-formaldehyde. Cell wall was partially digested with enzymes. Specific antibodies were introduced to transgenic cells. Extra unbound antibodies were washed. Chemi-fluorescent secondary antibody was introduced to cells. Excess secondary antibodies were washed. Cells were fixed on glass slide and examined under fluorescent confocal microscope. Microscopic fluorescence of BY2 Cells expressing different proteins was analyzed to reveal subcellular localization of the different target proteins. See FIG. 21 and FIG. 22. The results summarized in Table 1 indicate that the target protein of the present invention (e.g. PBSV-1 & 2). Is localized to PBs in a manner similar to other PB targeted proteins.

### Example 6

### Subcellular Localization of Mutations in Instant Constructs

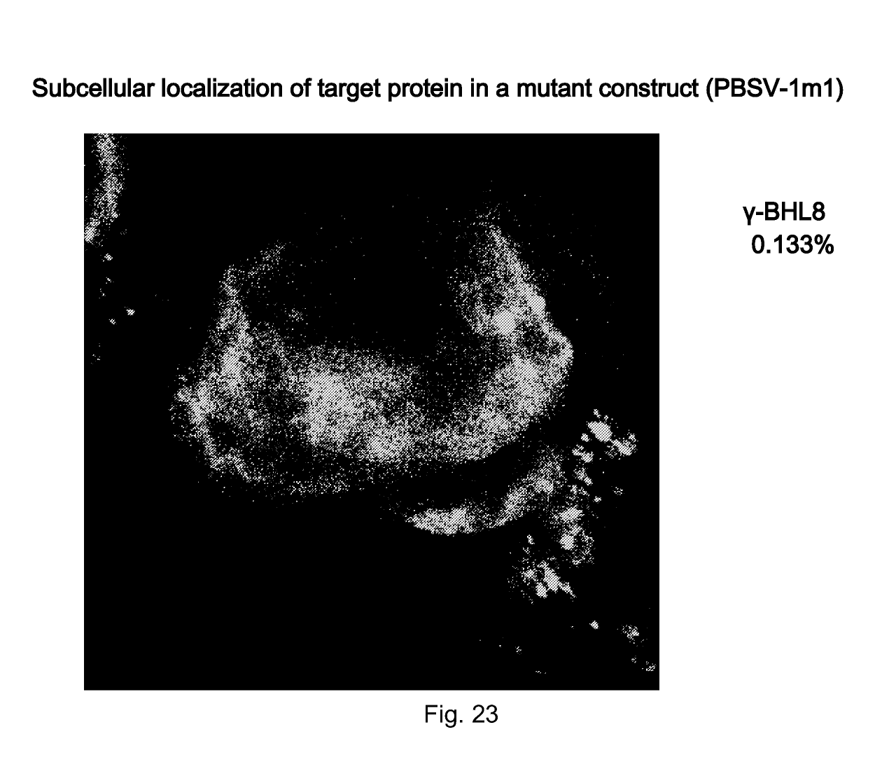

A mutant PBSV (PBSV-1 ml) was constructed such that it did not contain the TMD or the PPID sequences. PBSV-1 and PBSV-1 ml were transformed into BY2 cells to further characterize the role of certain elements of the present invention in subcellular localization. As shown in FIG. 23, the target protein construct without the TMD and PPID (in PBSV-1 ml) properly become localized to the ER but do not form protein bodies, demonstrating the importance of these technical features (i.e. TMD and PPID) of the present invention. Using this model system, the skilled artisan can now determine desirable sequences—i.e. sequences that favorably result in PB localization.

### Example 7

### The Role of CID in Subcellular Localization of Target Proteins

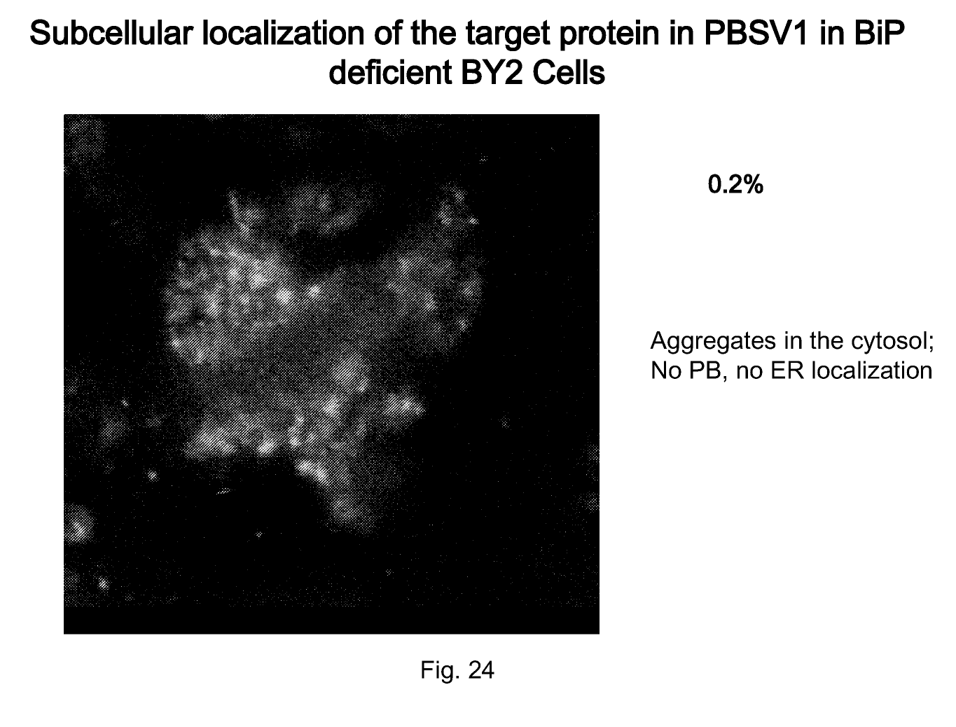

PBSV-1 was transformed into BY2 cells deficient (by knock-out) of BIP, the ER-luminal chaperonin in the host plant. As shown in FIG. 24, the target protein is not observed in the ER. However, the protein is still functional in forming aggregates.

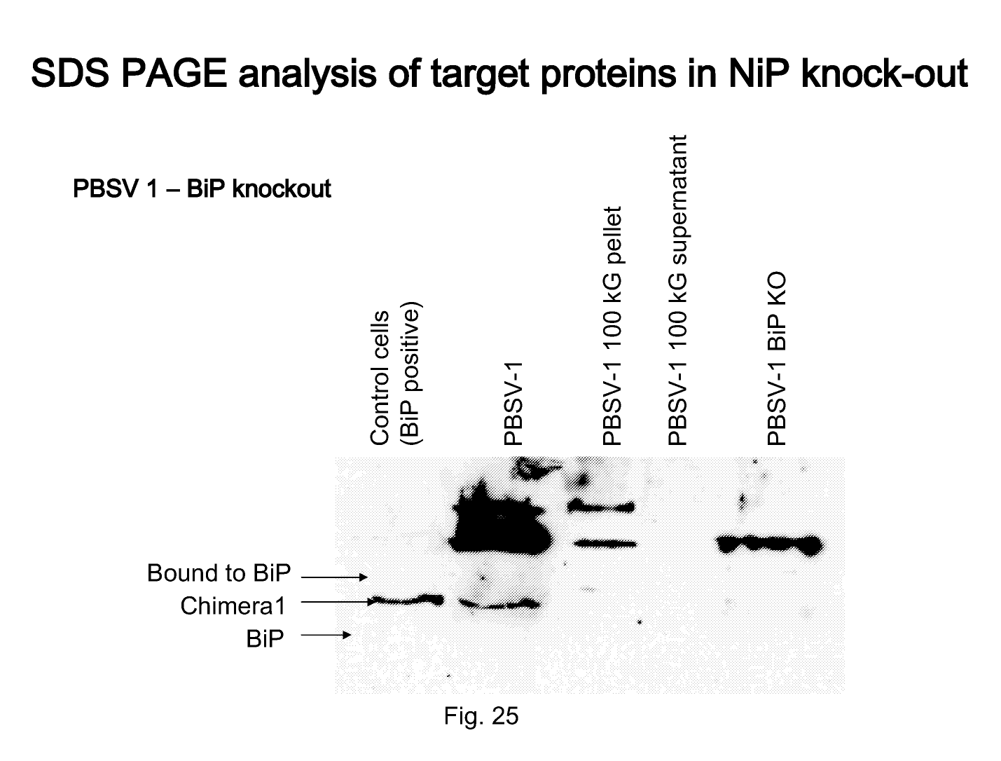

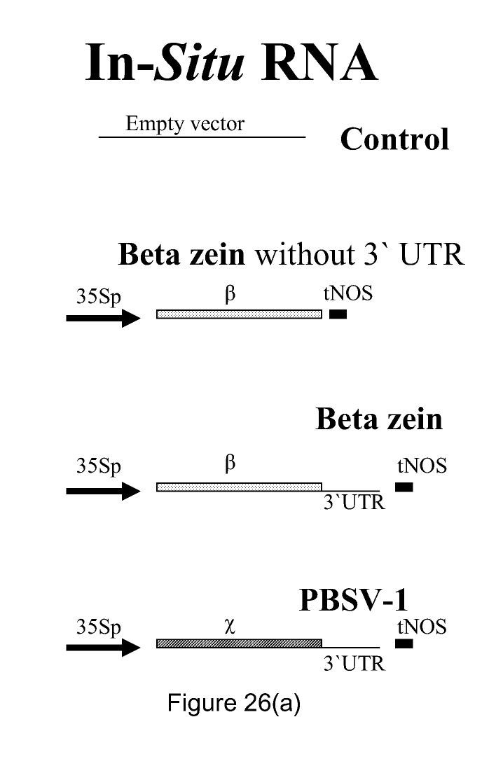

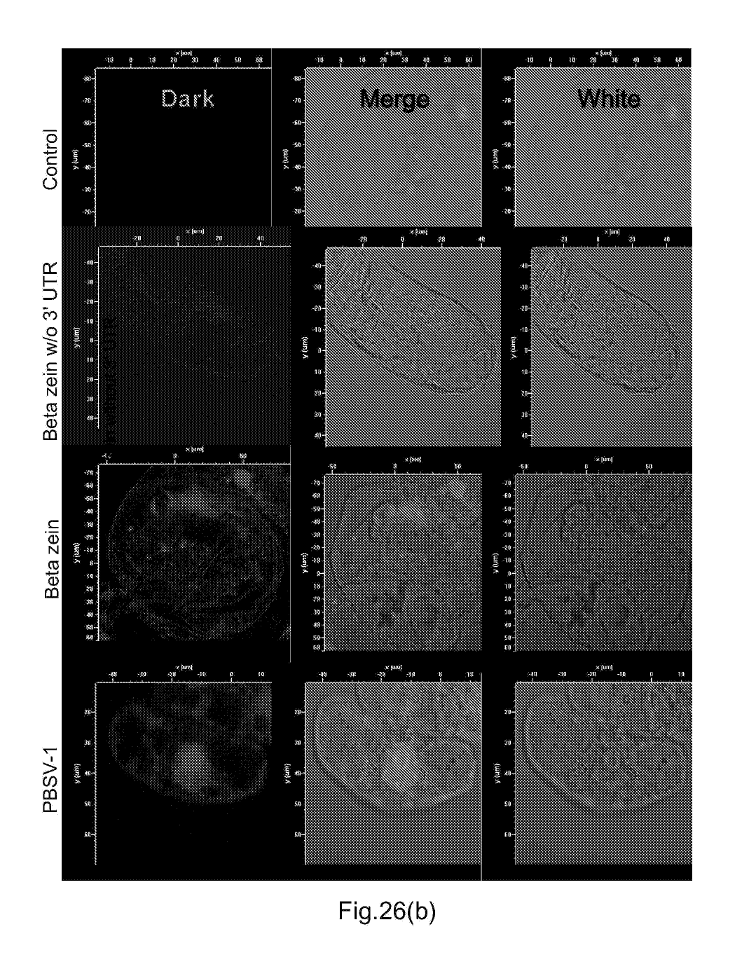

As shown in FIG. 25, SDS-PAGE analysis shows that PBSV-1 transformed in a BiP knock out fails to bind to BiP and fails to become localized in PBs. This example demonstrates the role of the CID in the present invention.

### Example 8

### Analysis of RNA Production and Stability for Protein Products Produced in Plants Transformed by Present Constructs

This experiment demonstrates the role of 3′UTR in stabilizing the RNA transcript in transgenic BY2 cell lines. Construct having 3′UTR were compared with same constructs without 3′UTR through RNA localization by in-situ hybridization. Cells were fixed and cell wall partially lysed then hybridized with Cy3 labeled probes and examined under the confocal microscope. Cy3 dye is a fluorescent dye when excited by laser wavelength of 488 nm. As shown in Figure, the 3′UTR stabilizes transcript level and increasing its translation. Thus, TED sequences are useful with present constructs to increase accumulation of target protein.

### Example 9

### Analysis of GFP Target Protein Production from a Constructs of the Present Invention in Transformed Plants

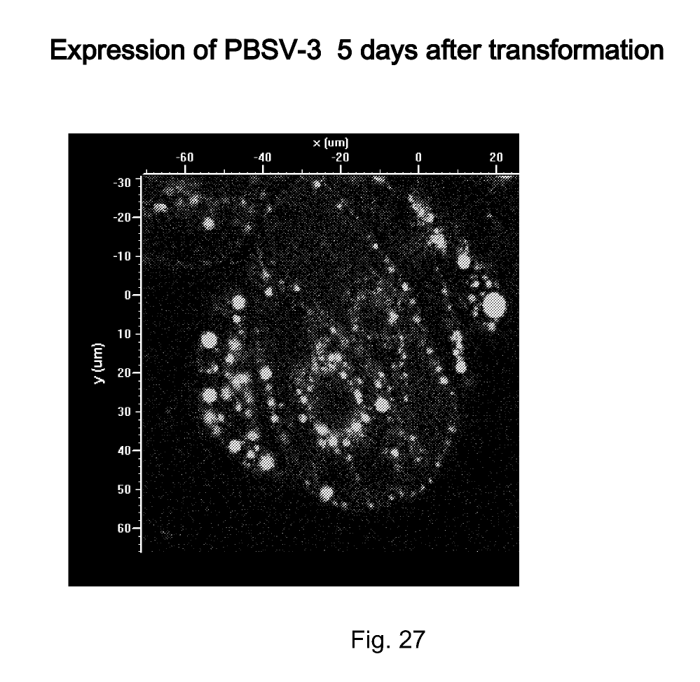

PBSV-3 was expressed in BY2 cells and the subcellular localization of the target protein (GFP) was examined by GFP florescence. As shown in FIG. 27, GFP accumulates in protein bodies. Thus, the present invention is useful for a wide range of target proteins (e.g. those having enzymatic activity and those that having structural or chemical purposes).

### Example 10

### Orientation of the CID, TMD and PPID Sequences Upstream to the Target Sequence Results in PB Formation

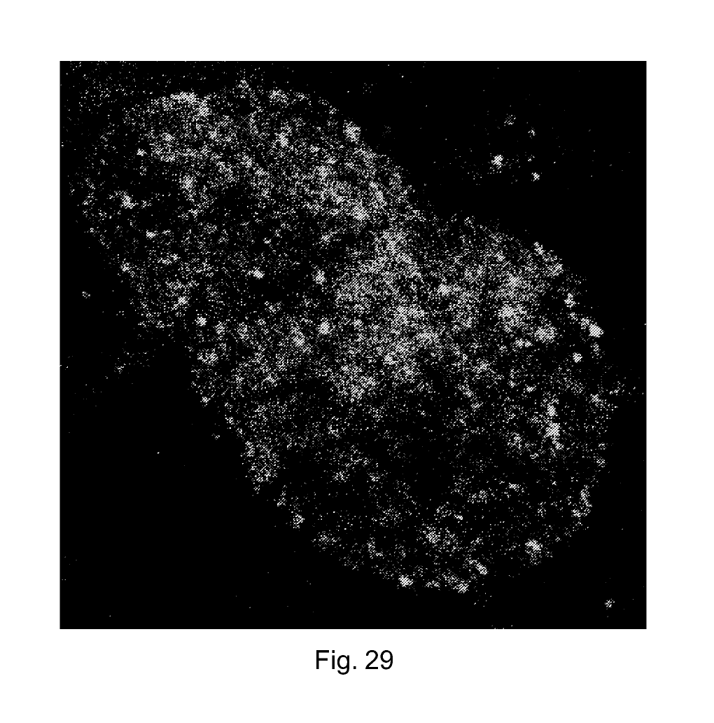

PBSV-4 was reconstructed to have the CID (SEQ ID No: 3), TMD (SEQ ID No: 1) and PPID (SEQ ID No: 2) sequences upstream of the Sporamine target protein sequence as shown in FIG. 28 The PBSV-4 nucleic acid sequence is provided as SEQ ID NO:19 while the protein encoded by PBSV-4 is provided as SEQ ID NO:20. The resulting construct was transformed into tobacco BY2 cells by Agrobacterium mediated transformation. The transformed cells were fixed and expressed protein were immunolocalized by hybridizing to specific primary antibodies and fluorescent secondary antibodies, which later was visualized under the confocal microscope as shown in FIG. 29.

As shown in FIG. 28, the PB were formed in transgenic BY2 cells by fusing the target protein to the CID, TMD and PPID to the N-terminal end of the target protein. This example demonstrates that CID, TMD and PPID are essential elements to form PB and furthermore, can be placed upstream of the target protein for more convenient cloning purposes.

### Example 11

### Protein Purification by Ficoll Gradient from Transgenic Cells

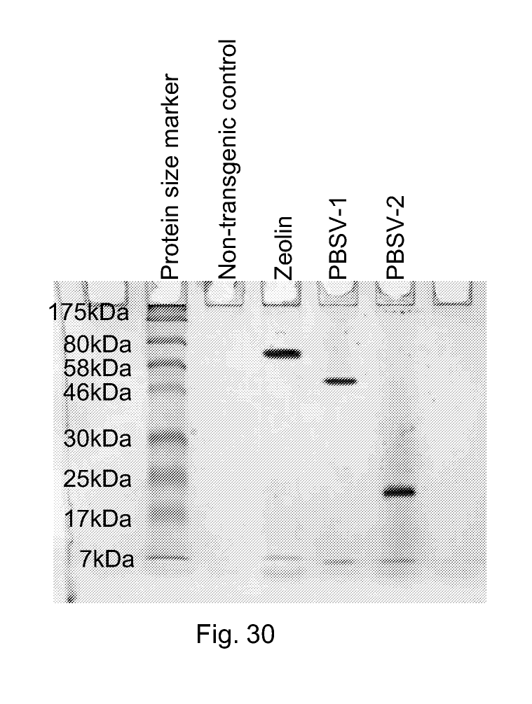

Transgenic cassava expressing Zeolin, PBSV-1 or PBSV-2 were used to extract and purify the target protein. Cassava storage roots were washed and peeled then homogenized in extraction buffer and filtrated through four layers of cheesecloth. The crude protein extract was placed on a layer of 15% ficoll cushion and centrifuged at 4,000 rpm for 15 minutes. PB's precipitate at the bottom of the centrifuge tube, where it could be collected by discarding the liquid layer and washing with water. The purified protein could be stored in glycerol. The exact expected size of the protein is shown in FIG. 30, where the purified proteins were loaded on 12% SDS-PAGE and stained with Coomassie-blue protein stain.

As various modifications could be made in the constructions and methods herein described and illustrated without departing from the scope of the invention, it is intended that all matter contained in the foregoing description or shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

1. A construct comprising a promoter functional in a host and a heterologous polynucleotide comprising:
(a) a sequence that codes for a moiety that interacts with an ER luminal folding chaperone interacting domain (CID);
(b) a sequence that codes for a trans-membrane domain (TMD); and
(c) a sequence that codes for a protein-protein interaction domain (PPID),
wherein the heterologous polynucleotide is operably linked to the promoter.

2. The construct of claim 1, wherein said heterologous polynucleotide does not encode a naturally occurring form of a protein sequence comprising all of said sequences (a), (b), and (c).

3. The construct of claim 1, wherein said heterologous polynucleotide comprises a site for operable insertion of a sequence that encodes a polypeptide.

4. The construct of claim 3, wherein said heterologous polynucleotide comprises in 5′ to 3′ order: (i) said sequence of (a); (ii) said site for operable insertion of a sequence; (iii) said sequence of (b); and (iv) said sequence of (c), wherein insertion of a sequence that encodes a polypeptide in said site for operable insertion provides for operable linkage of said sequence encoding said polypeptide to (i), (iii), and (iv).

5. The construct of claim 3, wherein said heterologous polynucleotide comprises in 5′ to 3′ order: (i) said sequence of (a); (ii) said sequence of (b); (iii) said sequence of (c); and (iv) said site for operable insertion of a sequence, wherein insertion of a sequence that encodes a polypeptide in said site for operable insertion provides for operable linkage of said sequence encoding said polypeptide to (i), (ii), and (iii).

6. The construct of claim 1, wherein the heterologous polynucleotide further comprises a sequence coding for a target protein that is operably linked to: i) said sequence that codes for a chaperone interacting domain (CID); ii) said sequence that codes for a trans-membrane-domain (TMD); and iii) said sequence that codes for a protein-protein interaction domain (PPID).

7. The construct of claim 6, wherein said heterologous polynucleotide comprises in 5′ to 3′ order: (i) said sequence of (a); (ii) said sequence encoding a target protein; (iii) said sequence of (b); and (iv) said sequence of (c), wherein (i), (ii), (iii), and (iv) are operably linked.

8. The construct of claim 6, wherein said heterologous polynucleotide comprises in 5′ to 3′ order: (i) said sequence of (a); (ii) said sequence of (b); (iii) said sequence of (c); and (iv) said sequence encoding a target protein; wherein (i), (ii), (iii), and (iv) are operably linked.

9. The construct of claim 6, wherein said target protein is BHL8, Sporamine, or a Green Fluorescent Protein (GFP).

10. The construct of claim 6, wherein said target protein does not in a naturally occurring form comprise any one of or all of: (a) a sequence that codes for a moiety that interacts with an ER luminal folding chaperone interacting domain (CID); (b) a sequence that codes for a trans-membrane-coding domain (TMD); and (c) a sequence that codes for a protein-protein interaction domain (PPID).

11. The construct of claim 1, further comprising at least one operably linked regulatory element selected from a 3′ UTR translational enhancer domain and a terminator.

12. A host transformed by the construct of claim 1.

13. The host of claim 12, wherein the host accumulates the target protein in protein bodies.

14. The host of claim 12 wherein the target protein is substantially non-glycosylated.

15. The host of claim 12, wherein the host is a cell, a tissue, an organ, or non-human organism.

16. The host of claim 12, wherein the host is a monocot plant, a monocot plant cell, a monocot plant seed, a dicot plant, a dicot plant seed, a dicot plant cell, a non-human animal, a procaryotic cell, or a eukaryotic cell.

17. (canceled)

18. A method of producing a target protein comprising growing a host with a construct of claim 1 under conditions that provide for expression of a protein encoded by said heterologous polynucleotide of said construct.

19. The method of claim 18, wherein said target protein does not form protein bodies in its naturally occurring form.

20. The method of claim 18, wherein said method further comprises purification of said protein encoded by said heterologous polynucleotide or purification of a portion of said protein encoded by said heterologous polynucleotide.

21. The method of claim 20, wherein said purification comprises a step wherein said protein or a portion thereof is recovered as a protein body.

22.-27. (canceled)


SEQUENCE LISTING (21 sequences)

SEQ ID NO: 1 - protein, 17 aa, Zea mays
VIVLVVCLALSAASASA

SEQ ID NO: 2 - protein, 158 aa, Zea mays
MQMPCPCAGLQGLYGAGAGLTTMMGAGGLYPYAEYLRQPQCSPLAAAPYYAGCGQPSAMFQPLRQQCCQQQMRMMDVQSVAQQLQMMMQLERAAAASSSLYEPALMQQQQQLLAAQGLNPMAMMMAQNMPAMGGLYQYQLPSYRTNPCGVSAAIPPYY

SEQ ID NO: 3 - protein, 49 aa, Zea mays
PPPPVHLPPPVHLPPPVHLPPPVHLPPPVHLPPPVHLPPPVHVPPPVHL

SEQ ID NO: 4 - protein, 24 aa, Arabidopsis thaliana
MKIMMMIKLCFFSMSLICIAPADA

SEQ ID NO: 5 - protein, 24 aa, Oryza sativa
MAASHGNAIFVLLLCTLFLPSLAC

SEQ ID NO: 6 - protein, 24 aa, Arabidopsis thaliana
MAARIGIFSVFVAVLLSISAFSSA

SEQ ID NO: 7 - protein, 30 aa, Arabidopsis thaliana
MTNMTLRKHVIYFLFFISCSLSKPSDASRG

SEQ ID NO: 8 - protein, 457 aa, Artificial Sequence
  Synthetic
MCGCQPPPPVHLPPPVHLPPPVHLPPPVHLPPPVHLPPPVHLPPPVHVPPPVHLPPPPCHMKAFTLALFLALFLYLLPNPAHSRFNPIRLPTTHEPASSETPVLDINGDEVRAGGNYYMVSAIWGAGGGGLRLAHLDTMSKCASDVIVSPNDLDNGDPITITPATADPESTVVMASTYQTFRFNIANNKLCVKNVNWGIQHDSASGQYFLKDGEFVSDNSNQFKIEVVDANLNFYKLTYCQFGSDKCYNCGRFHDPMLRTTRLALSNSPFVFVIKPTDVCKMVIVLVVCLALSAASASAMQMPCPCAGLQGLYGAGAGLTTMMGAGGLYPYAEYLRQPQCSPLAAAPYYAGCGQPSAMFQPLRQQCCQQQMRMMDVQSVAQQLQMMMQLERAAAASSSLYEPALMQQQQQLLAAQGLNPMAMMMAQNMPAMGGLYQYQLPSYRTNPCGVSAAIPPYY

SEQ ID NO: 9 - protein, 457 aa, Artificial Sequence; identical to SEQ ID NO: 8
  Synthetic

SEQ ID NO: 10 - protein, 514 aa, Artificial Sequence
  Synthetic
MCGCQPPPPVHLPPPVHLPPPVHLPPPVHLPPPVHLPPPVHLPPPVHVPPPVHLPPPPCHMKTNLFLFLIFSLLLSLSSAEFAMEYPYDVPDYASEILGGGSKGEELFTGVVPILVELDGDVNGHKFSVSGEGEGDATYGKLTLKFICTTGKLPVPWPTLVTTFSYGVQCFSRYPDHMKRHDFFKSAMPEGYVQERTIFFKDDGNYKTRAEVKFEGDTLVNRIELKGIDFKEDGNILGHKLEYNYNSHNVYIMADKQKNGIKANFKTRHNIEDGGVQLADHYQQNTPIGDGPVLLPDNHYLSTQSALSKDPNEKRDHMVLLEFVTAAGITHGMDELMKMVIVLVVCLALSAASASAMQMPCPCAGLQGLYGAGAGLTTMMGAGGLYPYAEYLRQPQCSPLAAAPYYAGCGQPSAMFQPLRQQCCQQQMRMMDVQSVAQQLQMMMQLERAAAASSSLYEPALMQQQQQLLAAQGLNPMAMMMAQNMPAMGGLYQYQLPSYRTNPCGVSAAIPPYY

SEQ ID NO: 11 - protein, 287 aa, Artificial Sequence
  Synthetic
MPPPVHLPPPVHLPPPVHLPPPVHLPPPVHLPPPVHLPPPVHVPPPVHLVIVLVVCLALSAASASASRMKAFTLALFLALFLYLLPNPAHSRFNPIRLPTTHEPASSETPVLDINGDEVRAGGNYYMVSAIWGAGGGGLRLAHLDTMSKCASDVIVSPNDLDNGDPITITPATADPESTVVMASTYQTFRFNIANNKLCVKNVNWGIQHDSASGQYFLKDGEFVSDNSNQFKIEVVDANLNFYKLTYCQFGSDKCYNCGRFHDPMLRTTRLALSNSPFVFVIKPTDV

SEQ ID NO: 12 - DNA, 141 bp, Artificial Sequence
  Synthetic
tgattcatgatatttgggaaatctcctctatccatctctctctatctatatatgtaataatgcagtaagacgacacacattatcatgtgtggtatgaccaataatatatgcatggtcataataaagttttggttttaaaaa

SEQ ID NO: 13 - protein, 525 aa, Artificial Sequence
  Synthetic
MMRARVPLLLLGILFLASLSASFATSLREEEESQDNPFYFNSDNSWNTLFKNQYGHIRVLQRFDQQSKRLQNLEDYRLVEFRSKPETLLLPQQADAELLLVVRSGSAILVLVKPDDRREYFFLTSDNPIFSDHQKIPAGTIFYLVNPDPKEDLRIIQLAMPVNNPQIHEFFLSSTEAQQSYLQEFSKHILEASFNSKFEEINRVLFEEEGQQEGVIVNIDSEQIKKLSKHAKSSSRKSLSKQDNTIGNEFGNLTERTDNSLNVLISSIEMEEGALFVPHYYSKAIVILVVNEGEAHVELVGPKGNKETLEYESYRAELSKDDVFVIPAAYPVAIKATSNVNFFGFGINANNNNRNLLAGKTDNVISSIGRALDGKDVLGLTFSGSGDEVMKLINKQSGSYFVDAHHHQQEQQKGRKGAFVYGGGGSGGGGSGGGGSGGCGCQPPPPVHLPPPVHLPPPVHLPPPVHLPPPVHLPPPVHLPPPVHVPPPVHLPPPPCHYPTQPPRPQPHPQPHPCPCQQPHPSPCQ

SEQ ID NO: 14 - DNA, 1371 bp, Artificial Sequence
  Synthetic
atgtgcggctgccagccaccgccgccggttcatctaccgccgccggtgcatctgccacctccggttcacctgccacctccggtgcatctcccaccgccggtccacctgccgccgccggtccacctgccaccgccggtccatgtgccgccgccggttcatctgccgccgccaccatgccacatgaaagccttcacactcgctctcttcttagctcttttcctctatctcctgcccaatccagcccattccaggttcaatcccatccgcctccccaccacacacgaacccgcctcctctgaaactccagtactcgacatcaacggcgacgaggtccgcgccggcgggaactactacatggtctccgccatatggggagccggcgggggagggctaagactcgcccacttggacacgatgtccaaatgcgccagcgacgtcatcgtatcccccaacgacttagacaacggcgaccccatcaccatcacgccggcgacggccgacccggaatccaccgtggtcatggcgtcgacctaccagactttccggttcaatatcgccaacaacaaactgtgcgtgaagaacgtgaactggggaatccagcacgacagcgcgtccgggcagtatttcctgaaagacggcgagtttgtctccgacaatagcaaccagttcaagattgaggtggtggatgccaaccttaacttctacaaactcacttactgtcagttcggctccgacaaatgctacaactgcggcagattccacgaccccatgttgaggaccacgcgcttggctctctccaattctcccttcgtttttgtcatcaaacctaccgatgtgtgcaagatggtcatcgttctcgtcgtgtgcctggctctgtcagctgccagcgcctctgcaatgcagatgccctgcccctgcgcggggctgcagggcttgtacggcgctggcgccggcctgacgacgatgatgggcgccggcgggctgtacccctacgcggagtacctgaggcagccgcagtgcagcccgctggcggcggcgccctactacgccgggtgtgggcagccgagcgccatgttccagccgctccggcaacagtgctgccagcagcagatgaggatgatggacgtgcagtccgtcgcgcagcagctgcagatgatgatgcagcttgagcgtgccgctgccgccagcagcagcctgtacgagccagctctgatgcagcagcagcagcagctgctggcagcccagggtctcaaccccatggccatgatgatggcgcagaacatgccggccatgggtggactctaccagtaccagctgcccagctaccgcaccaacccctgtggcgtctccgctgccattccgccctactac

SEQ ID NO: 15 - DNA, 1542 bp, Artificial Sequence
  Synthetic
atgtgcggctgccagccaccgccgccggttcatctaccgccgccggtgcatctgccacctccggttcacctgccacctccggtgcatctcccaccgccggtccacctgccgccgccggtccacctgccaccgccggtccatgtgccgccgccggttcatctgccgccgccaccatgccacatgaagactaatctttttctctttctcatcttttcacttctcctatcattatcctcggccgaattcgccatggaatacccgtacgacgttccggactacgcttctgagatcttaggtgggggtagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttctcttatggtgttcaatgcttttcaagatacccagatcatatgaagcggcacgacttcttcaagagcgccatgcctgagggatacgtgcaggagaggaccatcttcttcaaggacgacgggaactacaagacacgtgctgaagtcaagtttgagggagacaccctcgtcaacaggatcgagcttaagggaatcgatttcaaggaggacggaaacatcctcggccacaagttggaatacaactacaactcccacaacgtatacatcatggccgacaagcaaaagaacggcatcaaagccaacttcaagacccgccacaacatcgaagacggcggcgtgcaactcgctgatcattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactaatgaagatggtcatcgttctcgtcgtgtgcctggctctgtcagctgccagcgcctctgcaatgcagatgccctgcccctgcgcggggctgcagggcttgtacggcgctggcgccggcctgacgacgatgatgggcgccggcgggctgtacccctacgcggagtacctgaggcagccgcagtgcagcccgctggcggcggcgccctactacgccgggtgtgggcagccgagcgccatgttccagccgctccggcaacagtgctgccagcagcagatgaggatgatggacgtgcagtccgtcgcgcagcagctgcagatgatgatgcagcttgagcgtgccgctgccgccagcagcagcctgtacgagccagctctgatgcagcagcagcagcagctgctggcagcccagggtctcaaccccatggccatgatgatggcgcagaacatgccggccatgggtggactctaccagtaccagctgcccagctaccgcaccaacccctgtggcgtctccgctgccattccgccctactac

SEQ ID NO: 16 - DNA, 862 bp, Artificial Sequence
  Synthetic
atgccgccgccggttcatctaccgccgccggtgcatctgccacctccggttcacctgccacctccggtgcatctcccaccgccggtccacctgccgccgccggtccacctgccaccgccggtccatgtgccgccgccggttcatctggtcatcgttctcgtcgtgtgcctggctctgtcagctgccagcgcctctgcatctagaatgaaagccttcacactcgctctcttcttagctcttttcctctatctcctgcccaatccagcccattccaggttcaatcccatccgcctccccaccacacacgaacccgcctcctctgaaactccagtactcgacatcaacggcgacgaggtccgcgccggcgggaactactacatggtctccgccatatggggagccggcgggggagggctaagactcgcccacttggacacgatgtccaaatgcgccagcgacgtcatcgtatcccccaacgacttagacaacggcgaccccatcaccatcacgccggcgacggccgacccggaatccaccgtggtcatggcgtcgacctaccagactttccggttcaatatcgccaacaacaaactgtgcgtgaagaacgtgaactggggaatccagcacgacagcgcgtccgggcagtatttcctgaaagacggcgagtttgtctccgacaatagcaaccagttcaagattgaggtggtggatgccaaccttaacttctacaaactcacttactgtcagttcggctccgacaaatgctacaactgcggcagattccacgaccccatgttgaggaccacgcgcttggctctctccaattctcccttcgtttttgtcatcaaacctaccgatgtgt

SEQ ID NO: 17 - DNA, 1516 bp, Artificial Sequence
  Synthetic
gcctcatttgccacttcactccgggaggaggaagagagccaagataaccccttctacttcaactctgacaactcctggaacactctattcaagaaccaatatggtcacattcgtgtcctccagaggttcgaccaacaatccaaacgacttcagaatcttgaagactaccgtcttgtggagttcaggtccaaacccgaaaccctccttcttcctcagcaggctgatgctgagttactcctagttgtccgtagtgggagcgccatactcgtcttggtgaaacctgatgatcgcagagagtacttcttccttacgagcgataacccgatattctctgatcaccagaaaatccctgcaggaaccattttctatttggttaaccctgatcccaaagaggatctcagaataatccaactcgccatgcccgttaacaaccctcagattcatgaatttttcctatctagcacagaagcccaacaatcctacttgcaagagttcagcaagcatattctagaggcctccttcaatagcaaattcgaggagatcaacagggttctgtttgaagaggagggacagcaagagggagtgattgtgaacattgattctgaacagattaagaaactgagcaaacatgcaaaatctagttcaaggaaatccctttccaaacaagataacacaattggaaacgaatttggaaacctgactgagaggaccgataactccttgaatgtgttaatcagttctatagagatggaagagggagctctttttgtgccacactactattctaaggccattgttatactagtggttaatgaaggagaagcacatgttgaacttgttggcccaaaaggaaataaggaaaccttggaatatgagagctacagagctgagctttctaaagacgatgtatttgtaatcccagcagcatatccagttgccatcaaggctacctccaacgtgaatttctttggtttcggtatcaatgctaataacaacaataggaacctccttgcaggtaagacggacaatgtcataagcagcatcggtagagctctggacggtaaagacgtgttggggcttacgttctctgggtctggtgacgaagttatgaagctgatcaacaaacagagtggatcgtactttgtggatgcacaccatcaccaacaggaacagcaaaagggaagaaagggtgcatttgtgtacggaggtgggggatccggagggggcggttcaggcggaggtggctcaggcggctgcggctgccagccaccgccgccggttcatctaccgccgccggtgcatctgccacctccggttcacctgccacctccggtgcatctcccaccgccggtccacctgccgccgccggtccacctgccaccgccggtccatgtgccgccgccggttcatctgccgccgccaccatgccactaccctactcaaccgccccggcctcagcctcatccccagccacacccatgcccgtgccaacagccgcatccaagcccgtgccagt

SEQ ID NO: 18 - protein, 6 aa, Zea mays
PPPVHL

SEQ ID NO: 19 - DNA, 877 bp, Artificial
  synthetic
atgccgccgccggttcatctaccgccgccggtgcatctgccacctccggttcacctgccacctccggtgcatctcccaccgccggtccacctgccgccgccggtccacctgccaccgccggtccatgtgccgccgccggttcatctggtcatcgttctcgtcgtgtgcctggctctgtcagctgccagcgcctctgcatgctgctgtaagtgctctagaatgaaagccttcacactcgctctcttcttagctcttttcctctatctcctgcccaatccagcccattccaggttcaatcccatccgcctccccaccacacacgaacccgcctcctctgaaactccagtactcgacatcaacggcgacgaggtccgcgccggcgggaactactacatggtctccgccatatggggagccggcgggggagggctaagactcgcccacttggacacgatgtccaaatgcgccagcgacgtcatcgtatcccccaacgacttagacaacggcgaccccatcaccatcacgccggcgacggccgacccggaatccaccgtggtcatggcgtcgacctaccagactttccggttcaatatcgccaacaacaaactgtgcgtgaagaacgtgaactggggaatccagcacgacagcgcgtccgggcagtatttcctgaaagacggcgagtttgtctccgacaatagcaaccagttcaagattgaggtggtggatgccaaccttaacttctacaaactcacttactgtcagttcggctccgacaaatgctacaactgcggcagattccacgaccccatgttgaggaccacgcgcttggctctctccaattctcccttcgtttttgtcatcaaacctaccgatgtgt

SEQ ID NO: 20 - protein, 292 aa, Artificial
  synthetic
MPPPVHLPPPVHLPPPVHLPPPVHLPPPVHLPPPVHLPPPVHVPPPVHLVIVLVVCLALSAASASACCCKCSRMKAFTLALFLALFLYLLPNPAHSRFNPIRLPTTHEPASSETPVLDINGDEVRAGGNYYMVSAIWGAGGGGLRLAHLDTMSKCASDVIVSPNDLDNGDPITITPATADPESTVVMASTYQTFRFNIANNKLCVKNVNWGIQHDSASGQYFLKDGEFVSDNSNQFKIEVVDANLNFYKLTYCQFGSDKCYNCGRFHDPMLRTTRLALSNSPFVFVIKPTDV

SEQ ID NO: 21 - DNA, 1014 bp, Artificial
  synthetic
atgtgcggctgccagccaccgccgccggttcatctaccgccgccggtgcatctgccacctccggttcacctgccacctccggtgcatctcccaccgccggtccacctgccgccgccggtccacctgccaccgccggtccatgtgccgccgccggttcatctgccgccgccaccatgccacggatccgccaccagcaagggcctcaagggtgtgatggtgtgtttacttatactggggttggttctcgaacaggtgcaagtagaaggcaagagtagcatggctaagatgaagtgcacgtggcctgagctggtgggcaagaccgtggagaaagccaagaagatgatcatgaaggacaagccagaggcgaagatcatggttctgccagttgggaccaaagtgaccggtgaatggaagatggatcgcgtccgcctctgggtcgacaagaaggacaagatcgccaagactccgaagtgcggcgaattcaagatggtcatcgttctcgtcgtgtgcctggctctgtcagctgccagcgcctctgcaatgcagatgccctgcccctgcgcggggctgcagggcttgtacggcgctggcgccggcctgacgacgatgatgggcgccggcgggctgtacccctacgcggagtacctgaggcagccgcagtgcagcccgctggcggcggcgccctactacgccgggtgtgggcagccgagcgccatgttccagccgctccggcaacagtgctgccagcagcagatgaggatgatggacgtgcagtccgtcgcgcagcagctgcagatgatgatgcagcttgagcgtgccgctgccgccagcagcagcctgtacgagccagctctgatgcagcagcagcagcagctgctggcagcccagggtctcaaccccatggccatgatgatggcgcagaacatgccggccatgggtggactctaccagtaccagctgcccagctaccgcaccaacccctgtggcgtctccgctgccattccgccctactac